(19) United States
(12) Patent Application Publication
(10) Pub. No.: US 2015/0017130 A1
(43) Pub. Date: Jan. 15, 2015

(54) METHODS AND COMPOSITIONS FOR INHIBITION OF IMMUNE RESPONSES

(71) Applicant: The General Hospital Corporation, Boston, MA (US)

(72) Inventors: Yongguang Yang, Cambridge, MA (US); Megan Sykes, Bronx, NY (US)

(21) Appl. No.: 14/297,357

(22) Filed: Jun. 5, 2014

Related U.S. Application Data
(63) Continuation of application No. 11/519,667, filed on Sep. 12, 2006.
(60) Provisional application No. 60/716,875, filed on Sep. 13, 2005.

(51) Int. Cl. A61K 38/17; A01K 67/027; C07K 14/705; A61K 35/12; C12N 15/85

(52) U.S. Cl. CPC A61K 38/1774; A61K 35/12; C12N 15/8509; C07K 14/70503; A01K 67/0275; A61K 48/00
USPC 424/93.7; 435/366; 435/325; 800/14; 800/17

(57) ABSTRACT

Methods and compositions for modulating immune responses are provided herein.

### CROSS-REFERENCE TO RELATED APPLICATIONS

This application is a continuation of U.S. Ser. No. 11/519,667, filed Sep. 12, 2006, which claims the benefit of priority of U.S. Ser. No. 60/716,875, filed Sep. 13, 2005, the contents of which are hereby incorporated by reference in their entirety for all purposes.

### TECHNICAL FIELD

This invention relates to methods and compositions for modulating immune responses, and more particularly to methods and compositions the inhibit graft rejection.

### BACKGROUND

CD47, known as integrin-associated protein, is a ubiquitously expressed 50-kDa cell surface glycoprotein and serves as a ligand for signal regulatory protein (SIRP)α (also known as CD172a, and SHPS-1). CD47 and SIRPα constitute a cell-cell communication system (the CD47-SIRPα system) that plays important roles in a variety of cellular processes including cell migration, adhesion of B cells, and T cell activation (Liu et al., J Biol Chem 277:10028, 2002; Motegi et al., Embo J 22:2634, 2003; Yoshida et al., J Immunol 168:3213, 2002; Latour et al., J Immunol 167:2547, 2001). In addition, the CD47-SIRPα system is implicated in negative regulation of phagocytosis by macrophages. CD47 on the surface of several cell types (i.e. erythrocytes, platelets or leukocytes) inhibits phagocytosis by macrophages.

The role of CD47/SIRPα interaction in the inhibition of phagocytosis has been illustrated by the observation that primary, wild-type mouse macrophages rapidly phagocytose unopsonized red blood cells (RBCs) obtained from CD47-deficient mice but not those from wild-type mice (Oldenborg et al., Science 288:2051, 2000). It has also been reported that through its receptors, SIRPα, CD47 inhibits both Fcγ and complement receptor mediated phagocytosis (Oldenborg et al., J Exp Med 193:855, 2001).

### SUMMARY

The activation of immune effector cells is regulated by inhibitory signals. The invention is based, in part, on the discovery that immune responses can be inhibited by manipulating the expression of ligands for inhibitory signaling molecules. In a cross-species transplant setting, certain ligands on donor cells do not efficiently interact with inhibitory receptors on host immune effector cells. Tolerance to xenogeneic cells may be promoted by expressing compatible (e.g., autologous) ligands for inhibitory molecules in the xenogeneic cells. For example, as demonstrated herein, CD47 molecules of certain species (e.g., swine CD47) fail to interact with SIRPα of other species (e.g., human SIRPα). Expression of human CD47 in swine cells renders the swine cells more resistant to immune recognition by human immune effector cells. The concept of manipulating ligand expression can be applied in additional ways to dampen undesirable immune reactions, as detailed further below.

Accordingly, in one aspect, the invention features a cell (e.g., an isolated cell, a purified cell, a cultured cell, a cell derived from a transgenic animal) of a first species comprising a nucleotide sequence (e.g., a transgene) encoding an immune-inhibitory molecule of a second species. In various embodiments, the immune-inhibitory molecule includes a CD47 polypeptide, or fragment or variant thereof, of a second species. Useful fragments and variants include those which retain the ability to bind with the appropriate receptor on an immune cell (e.g., a fragment which binds to SIRPα on a macrophage) and mediate at least one biological activity of the molecule (e.g., inhibition of phagocytosis, stimulation of tyrosine phosphorylation of SIRPα). For example, a cell which expresses the fragment or variant is less susceptible to phagocytosis by a phagocytic cell (e.g., a macrophage) of the second species, as compared to a control (e.g., a cell which does not express the fragment or variant).

In various embodiments, the immune-inhibitory molecule includes a sequence at least 80%, 85%, 90%, 95%, 96%, 97%, 98%, 99% identical to a human CD47 amino sequence, or a fragment thereof (e.g., the molecule has a sequence at least 80%, 85%, 90%, 95%, 96%, 97%, 98%, or 99% identical to the human CD47 amino sequence of SEQ ID NO:1, or a fragment thereof). In various embodiments, the immune-inhibitory molecule has a sequence which differs from the sequence of SEQ ID NO:1 in at least 1 amino acid position, but not more than 35 amino acid positions (e.g., the sequence differs from SEQ ID NO:1 at 1, 2, 3, 4, 5, 6, 7, 8, 9, 10, 11, 12, 13, 14, 15, 16, 17, 18, 19, 20, 21, 22, 23, 24, or 25 amino acid positions). The differences can be conservative and/or non-conservative amino acid substitutions.

Other suitable immune-inhibitory molecules are polypeptides which mediate inhibitory signals in immune cells (e.g., immune effector cells) and which interact less efficiently in a cross-species setting. For example, if a porcine ligand fails to interact, or interacts inefficiently, with a counterpart human receptor, the human form of the ligand is suitable for expression in a porcine cell. Ligands for macrophage inhibitory receptors with weak cross-species reactivity are contemplated. These include CD47, CD200, ligands for paired Ig-like receptor (PIR)-B, ligands for immunoglobulin-like transcript (ILT)3, and ligands for CD33-related receptors. Fragments and variants of these immune-inhibitory molecules are also contemplated. In various embodiments, the molecule is a molecule of a first species which, when expressed in a cell of a second species, renders the cell less susceptible to phagocytosis by a phagocytic cell of the first species.

In one embodiment, the first species is a non-human mammalian species (e.g., a swine species, a miniature swine species, or a non-human primate species).

In one embodiment, the cell is a cell of a transgenic animal, such as a germ cell line transgenic animal, e.g., a germ cell line transgenic miniature swine.

In one embodiment, the cell is a cell of a miniature swine which is at least partially inbred (e.g., the swine is homozygous at swine leukocyte antigen (SLA) loci, and/or is homozygous at at least 65%, 70%, 75%, 80%, 85%, 90%, 95%, or more, of all other genetic loci).

In one embodiment, the second species is human.

In various embodiments, the cell has been genetically modified (or is derived from a cell that has been genetically modified, e.g., the cell is a cell of a transgenic animal, such as a germ cell line transgenic animal) so as to include a second nucleotide sequence, e.g., encoding a second immune-inhibitory molecule of the second species, and/or a polypeptide of the second species. The polypeptide can be selected from an MHC polypeptide (e.g., an MHC class I polypeptide, an MHC class II polypeptide) and a complement regulatory protein (e.g., a CD55 polypeptide, a CD59 polypeptide, or a CD46 polypeptide).

In various embodiments, the cell has been genetically modified (or is derived from a cell that has been genetically modified) so as to be less reactive to natural antibodies of a second species. For example, the cell is deficient for expression of a carbohydrate modifying enzyme (e.g., α-1,3 galactosyltransferase), or expresses a carbohydrate modifying enzyme, such as an α-Galactosidase A (αGalA) enzyme.

The cell can be any type of cell. In various embodiments, the cell is a hematopoietic cell (e.g., a hematopoietic stem cell, lymphocyte, a myeloid cell), a pancreatic cell (e.g., a beta-islet cell), a kidney cell, a heart cell, or a liver cell.

In some embodiments, expression of the immune-inhibitory molecule (e.g., the CD47 polypeptide) is under the control of a heterologous promoter (e.g., a promoter that is endogenous to the first species). The promoter can be a tissue-specific promoter.

The invention also features a transgenic non-human mammal (e.g., a rodent, non-human primate, swine, cow, goat, or horse) whose genome includes a nucleotide sequence encoding a heterologous immune-inhibitory molecule (e.g., a CD47 polypeptide of a different species, such as a human CD47 polypeptide). In one embodiment, the mammal is a miniature swine.

The immune-inhibitory molecule (e.g., CD47 polypeptide) can be expressed in a cell and/or organ of the mammal in an amount sufficient to interact with a CD47 ligand such as signal regulatory protein a (SIRPα) on a different cell (e.g., on a human immune cell, such as a macrophage) and/or decrease immune recognition of the cell and/or organ by the different cell.

The invention also features an organ from a transgenic mammal of a first species whose genome comprises a nucleotide sequence encoding an immune-inhibitory molecule (e.g., a CD47 polypeptide) of a second mammalian species, wherein the organ expresses the immune-inhibitory molecule in an amount sufficient to decrease immune recognition of the organ by a cell of the second species. In various embodiments, the organ is a liver, a kidney, or a heart; the first species is a non-human mammalian species (e.g., a swine species, such as a miniature swine species); and the second species is human. The mammal from which the organ is derived can be genetically modified so as to further include a second nucleotide sequence, e.g., encoding a second immune-inhibitory molecule of the second species, and/or a polypeptide of the second species. The polypeptide can be selected from an MHC polypeptide (e.g., an MHC class I polypeptide, an MHC class II polypeptide), a complement regulatory protein (e.g., a CD55 polypeptide, a CD59 polypeptide, or a CD46 polypeptide), or a carbohydrate modifying enzyme, such as an α-Galactosidase A (αGalA) enzyme.

Alternatively, or in addition, the organ is deficient for expression of a carbohydrate modifying enzyme (e.g., α-1,3 galactosyltransferase).

In another aspect, the invention features a method for decreasing rejection of a graft in a host. The method includes, for example, increasing expression of an immune inhibitory molecule, such as CD47, in the graft. The graft can be an allograft (e.g., a graft from the same species as the host) or a xenograft.

In one embodiment, expression of the immune inhibitory molecule (e.g., CD47) is increased by expressing a transgene encoding the molecule. In one embodiment, the graft is a xenograft and the transgene encodes a CD47 polypeptide of the host species.

The invention also features a method of decreasing rejection of a graft in a host by administering an agent the binds to a receptor of an immune-inhibitory molecule in the host (e.g., an agent that binds to SIRPα, such as a soluble form of CD47 including all or a portion of the extracellular domain, e.g., an CD47-Fc, or an antibody that binds and activates signaling through SIRPα).

In another aspect, the invention features methods of supplying a graft. The methods include providing a donor graft, e.g., a kidney, liver, heart, thymus, hematopoietic stem cell, or pancreatic islet cell, wherein said graft expresses a heterologous immune-inhibitory molecule (e.g., CD47 polypeptide) or over express an endogenous immune-inhibitory molecule (e.g., CD47 polypeptide); and implanting said graft in a recipient; thereby supplying a graft. In various embodiments, the methods reduce hematopoietic-cell-mediated rejection of the graft and/or prolongs acceptance of the graft.

In various embodiments, the donor and recipient are of different species, e.g., the donor is a non-human animal, e.g., a miniature swine, and the recipient is a human. In some embodiments, the miniature swine graft expresses a human CD47, e.g., under the control of a heterologous promoter, and/or a constitutive promoter.

The method can include administering one or more treatments, e.g., a treatment which inhibits T cells, blocks complement, or otherwise down regulates the recipient immune response to the graft.

In one embodiment, the donor and recipient are of same species, e.g., they both are human, and expression of CD47 on the graft is upregulated.

The methods can include administration of one or more immunosuppressive agents (e.g., cyclosporine, FK506), antibodies (e.g., anti-T cell antibodies such as polyclonal anti-thymocyte antisera (ATG), and/or a monoclonal anti-human T cell antibody, such as LoCD2b), irradiation, and protocols to induce mixed chimerism.

In some embodiments, the recipient is thymectomized and/or splenectomized. Thymic irradiation can be used.

In some embodiments, the recipient is administered low dose radiation (e.g., a sublethal dose of between 100 rads and 400 rads whole body radiation).

The recipient can be treated with an agent that depletes complement, such as cobra venom factor.

Natural antibodies can be absorbed from the recipient's blood by hemoperfusion of a liver of the donor species. The cells, tissues, or organs used for transplantation may be genetically modified such that they are not recognized by natural antibodies of the host (e.g., the cells are α-1,3-galactosyltransferase deficient).

In some embodiments, the methods include treatment with a human anti-human CD154 mAb, mycophenolate mofetil, and/or methylprednisolone. The methods can also include agents useful for supportive therapy such as anti-inflammatory agents (e.g., prostacyclin, dopamine, ganiclovir, levofloxacin, cimetidine, heparin, antithrombin, erythropoietin, and aspirin).

In some embodiments, donor stromal tissue is administered.

The invention also features a breeding population of transgenic non-human mammals (e.g., rodents, non-human primates, swine, or cows) whose genomes comprise a nucleotide sequence encoding a human immune-inhibitory molecule (e.g., a human CD47 polypeptide), wherein a breeding population includes at least one male and one female.

The genomes can further include a nucleotide sequence encoding a second human polypeptide (e.g., a polypeptide selected from an MHC polypeptide (e.g., an MHC class I polypeptide, an MHC class II polypeptide), a complement regulatory protein (e.g., a CD55 polypeptide, a CD59 polypeptide, or a CD46 polypeptide), or a carbohydrate modifying enzyme, such as an α-Galactosidase A (αGalA) enzyme.

In various embodiments, the genomes are genetically altered such that a gene encoding a carbohydrate modifying enzyme (e.g., α-1,3 galactosyltransferase) has been inactivated.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a protein is preferably replaced with another amino acid residue from the same side chain family. Alternatively, in another embodiment, mutations can be introduced randomly along all or part of a coding sequence, such as by saturation mutagenesis, and the resultant mutants can be screened for biological activity to identify mutants that retain activity. Following mutagenesis, the encoded protein can be expressed recombinantly and the activity of the protein can be determined.

As used herein, a “biologically active portion” or a “functional domain” of a protein includes a fragment of a protein of interest which participates in an interaction, e.g., an intramolecular or an inter-molecular interaction, e.g., a binding or catalytic interaction. An inter-molecular interaction can be a specific binding interaction or an enzymatic interaction (e.g., the interaction can be transient and a covalent bond is formed or broken). An inter-molecular interaction can be between the protein and another protein, between the protein and another compound, or between a first molecule and a second molecule of the protein (e.g., a dimerization interaction). Biologically active portions/functional domains of a protein include peptides comprising amino acid sequences sufficiently homologous to or derived from the amino acid sequence of the protein which include fewer amino acids than the full length, natural protein, and exhibit at least one activity of the natural protein. Biological active portions/functional domains can be identified by a variety of techniques including truncation analysis, site-directed mutagenesis, and proteolysis. Mutants or proteolytic fragments can be assayed for activity by an appropriate biochemical or biological (e.g., genetic) assay. In some embodiments, a functional domain is independently folded. Typically, biologically active portions comprise a domain or motif with at least one activity of a protein, e.g., CD47. An exemplary domain is the CD47 extracellular domain. A biologically active portion/functional domain of a protein can be a polypeptide which is, for example, 10, 25, 50, 100, 200 or more amino acids in length.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, 50%, 60%, 70%, 80%, 90%, 95% or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using the NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of Meyers and Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used.

Some polypeptides of the present invention can have an amino acid sequence substantially identical to an amino acid sequence described herein. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. Methods of the invention can include use of a polypeptide that includes an amino acid sequence that contains a structural domain having at least about 60%, or 65% identity, likely 75% identity, more likely 85%, 90%, 92%, 94%, 95%, 96%, 97%, 98% or 99% identity to a domain of a polypeptide described herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. Methods of the invention can include use of a nucleic acid that includes a region at least about 60%, or 65% identity, likely 75% identity, more likely 85%, 90%, 91%, 92%, 93%, 94%, 95%, 96%, 97%, 98% or 99% identity to a nucleic acid sequence described herein, or use of a protein encoded by such nucleic acid.

A “purified preparation of cells”, as used herein, refers to an in vitro preparation of cells. In the case cells from multicellular organisms (e.g., plants and animals), a purified preparation of cells is a subset of cells obtained from the organism, not the entire intact organism. In the case of unicellular microorganisms (e.g., cultured cells and microbial cells), it consists of a preparation of at least 10% and more preferably 50% of the subject cells.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

As used herein, the term “transgene” means a nucleic acid sequence (encoding, e.g., a CD47 molecule), which is partly or entirely heterologous, i.e., foreign, to the transgenic animal or cell into which it is introduced. A transgene can include one or more transcriptional regulatory sequences and any other nucleic acid, such as introns, that may be necessary for optimal expression of the selected nucleic acid, all operably linked to the selected nucleic acid, and may include an enhancer sequence.

As used herein, the term “transgenic cell” refers to a cell containing a transgene.

As used herein, a “transgenic animal” is any animal in which one or more, and preferably essentially all, of the cells of the animal includes a transgene. The transgene is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. Transgenic swine which include one or more transgenes encoding one or more molecules are within the scope of this invention. For example, a double or triple transgenic animal, which includes two or three transgenes can be produced.

As used herein, the term “germ cell line transgenic animal” refers to a transgenic animal in which the transgene genetic information exists in the germ line, thereby conferring the ability to transfer the information to offspring. If such offspring in fact possess some or all of that information then they, too, are transgenic animals.

As used herein, the term “operably linked” means that selected DNA, e.g., encoding a class I peptide, is in proximity with a transcriptional regulatory sequence, e.g., tissue-specific promoter, to allow the regulatory sequence to regulate expression of the selected DNA.

The term “genetically programmed” as used herein means to permanently alter the DNA, RNA, or protein content of a cell.

As used herein, the term “recombinant swine cells” refers to cells derived from swine, preferably miniature swine, which have been used as recipients for a recombinant vector or other transfer nucleic acid, and include the progeny of the original cell which has been transfected or transformed. Recombinant swine cells include cells in which transgenes or other nucleic acid vectors have been incorporated into the host cell's genome, as well as cells harboring expression vectors which remain autonomous from the host cell's genome.

As used herein, the term “transfection” means the introduction of a nucleic acid, e.g., an expression vector, into a recipient cell by nucleic acid-mediated gene transfer. “Transformation”, as used herein, refers to a process in which a cell's genotype is changed as a result of the cellular uptake of exogenous DNA or RNA, and, e.g. the transformed swine cell expresses human cell surface peptides.

As used herein, the term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/expression of nucleic acids to which they are linked. Vectors capable of directing the expression of genes to which they are operatively linked are referred to herein as “expression vectors”.

“Transcriptional regulatory sequence” is a generic term used throughout the specification to refer to DNA sequences, such as initiation signals, enhancers, and promoters, which induce or control transcription of protein coding sequences with which they are operably linked. In preferred embodiments, transcription of the recombinant gene is under the control of a promoter sequence (or other transcriptional regulatory sequence) which naturally controls the expression of the recombinant gene in humans, or which naturally controls expression of the corresponding gene in swine cells. In some embodiments, the transcription regulatory sequence causes hematopoietic-specific expression of the recombinant protein. The above embodiments not withstanding, it will also be understood that the recombinant gene can be under the control of transcriptional regulatory sequences different from those sequences naturally controlling transcription of the recombinant protein. Transcription of the recombinant gene, for example, can be under the control of a synthetic promoter sequence. The promoter that controls transcription of the recombinant gene may be of viral origin; examples are promoters sometimes derived from bovine herpes virus (BHV), Moloney murine leukemia virus (MLV), SV40, Swine vesicular disease virus (SVDV), and cytomegalovirus (CMV).

As used herein, the term “tissue-specific promoter” means a DNA sequence that serves as a promoter, i.e., regulates expression of a selected DNA sequence operably linked to the promoter, and which effects expression of the selected DNA sequence in specific cells, e.g., hematopoietic cells or in epithelial cells. Particularly useful promoter sequences for directing expression include: promoter sequences naturally associated with the recombinant gene (e.g., the recombinant human CD47 sequence); promoter sequences naturally associated with the homologous gene of the host species (e.g., swine); promoters which are active primarily in hematopoietic cells, e.g. in lymphoid cells, in erythroid cells, or in myeloid cells or in epithelial cells; the immunoglobulin promoter described by Brinster et al. (1983) Nature 306:332-336 and Storb et al. (1984) Nature 310:238-231; the immunoglobulin promoter described by Ruscon et al. (1985) Nature 314:330-334 and Grosscheld et al. (1984) Cell 38:647-658; the globin promoter described by Townes et al. (1985) Mol. Cell. Biol. 5:1977-1983, and Magram et al. (1989) Mol. Cell. Biol. 9:4581-4584. Other promoters are described herein or will be apparent to those skilled in the art. Moreover, such promoters also may include additional DNA sequences that are necessary for expression, such as introns and enhancer sequences. The term also covers so-called “leaky” promoters, which regulate expression of a selected DNA primarily in one tissue, but cause expression in other tissues as well. Other regulatory elements e.g., locus control regions, e.g., DNase I hypersensitive sites, can be included.

By “cell specific expression”, it is intended that the transcriptional regulatory elements direct expression of the recombinant protein in particular cell types, e.g., bone marrow cells or epithelial cells.

“Graft”, as used herein, refers to a body part, organ, tissue, or cells. Grafts may consist of organs such as liver, kidney, heart or lung; body parts such as bone or skeletal matrix; tissue such as skin, intestines, endocrine glands; or progenitor stem cells of various types.

The term “tissue” as used herein, means any biological material that is capable of being transplanted and includes organs (especially the internal vital organs such as the heart, lung, liver, kidney, pancreas and thyroid), cornea, skin, blood vessels and other connective tissue, cells including blood and hematopoietic cells, Islets of Langerhans, brain cells and cells from endocrine and other organs and bodily fluids, all of which may be candidate for transplantation.

“A discordant species combination”, as used herein, refers to two species in which hyperacute rejection occurs when a graft is grafted from one to the other. In the subject invention, the donor is of porcine origin and the recipient is human.

“Hematopoietic stem cell”, as used herein, refers to a cell, e.g., a bone marrow cell, a fetal or neonatal liver or spleen cell, or a cord blood cell which is capable of developing into a mature myeloid and/or lymphoid cell.

“Progenitor cell”, as used herein, refers to a cell which gives rise to an differentiated progeny. In contrast to a stem cell, a progenitor cell is not always self renewing and is relatively restricted in developmental potential.

“Stromal tissue”, as used herein, refers to the supporting tissue or matrix of an organ, as distinguished from its functional elements or parenchyma.

“Tolerance”, as used herein, refers to the inhibition of a graft recipient's immune response which would otherwise occur, e.g., in response to the introduction of a nonself antigen into the recipient. Tolerance can involve humoral, cellular, or innate responses, or combinations thereof. Tolerance, as used herein, refers not only to complete immunologic tolerance to an antigen, but to partial immunologic tolerance, i.e., a degree of tolerance to an antigen which is greater than what would be seen if a method or composition described herein were not employed.

“Miniature swine”, as used herein, refers to wholly or partially inbred animal.

“Lymph node or thymic T cell”, as used herein, refers to T cells which are resistant to inactivation by traditional methods of T cell inactivation, e.g., inactivation by a single intravenous administration of anti-T cell antibodies, e.g., antibodies, e.g., ATG preparation.

The details of one or more embodiments of the invention are set forth in the accompanying drawings and the description below. Other features, objects, and advantages of the invention will be apparent from the description and drawings, and from the claims. All cited patents, patent applications, and references (including references to public sequence database entries) are incorporated by reference in their entireties for all purposes. U.S. Ser. No. 60/716,875 is incorporated by reference in its entirety for all purposes.

### DESCRIPTION OF DRAWINGS

FIG. 1A is a photograph depicting the results of Western blot analysis of SIRPα tyrosine phosphorylation in WT mouse macrophages. Macrophages were incubated in medium alone (Control; lane 1), or with CD47−/− mouse (lane 2), WT mouse (lane 3) or porcine (lane 4) RBCs for 30 min. Rows 1-2, Macrophage lysates were used directly in Western blot with anti-β-actin (row 1, as a loading control) or with anti-phosphotyrosine Ab (α-pTyr; row 2). Row 3, Macrophage lysates were immunoprecipitated by anti-SIRPα mAb P84; precipitated proteins were then analyzed by Western blot with anti-phosphotyrosine Ab (α-pTyr). A representative experiment of three is shown.

FIG. 1B is a graph depicting the results of experiments in which phagocytosis of porcine cells in the presence of SIRPα blocking antibodies was examined. Blocking SIRPα by anti-SIRPα mAb (P84) augments phagocytosis of WT mouse, but not CD47−/− mouse or porcine, RBCs. CFSE (green)-labeled splenic macrophages (5×10⁵/well) were incubated with or without anti-SIRPα antibody (P84) in 96-well plate for 20 minutes; then PKH-26 (red)-stained WT mouse (WT), CD47 KO mouse (CD47−/−), untreated pig (pRBC), or opsonized pig (ops pRBC) RBCs (1×10⁶/well) were added and phagocytosis was determined 1 hour after incubation using fluorescent microscope (engulfment was seen as a yellow event). The percent of macrophages engulfing target cells per well was calculated as follows: [number of yellow events/(number of yellow events+number of green non-engulfing macrophages)]×100%. Data are presented as mean±SDs (n=10-12 wells per group). ** p<0.01.

FIGS. 2A-2D are graphs depicting the results of experiments in which the clearance of cells injected into mice was examined. For FIGS. 2A-B, PKH26-labeled WT and CFSE-labeled CD47 KO mouse spleen cells were mixed at a ratio of 1:1, and intravenously injected into WT (A; n=3) or CD47 KO (B; n=3) mice (total 5×10⁷ per mouse). Mice were bled at 2, 8, 24, 48, and 72 hours after cell infusion, and the percentages of injected cells in WBCs were determined by flow cytometric analysis. Data shown are percentages (mean±SDs) of injected WT (•) and CD47 KO (∘) splenocytes, which were normalized with the levels at 2 hour after cell transfer as 100%. For FIGS. 2C-D, PKH26-labeled WT and CFSE-labeled CD47 KO mouse spleen cells were mixed at a ratio of 1:1, and intravenously injected into WT mice (total 5×10⁷ per mouse; n=3). Mice were bled at 2, 4, 8, 24, and 48 hours after cell infusion; WBCs were prepared and stained with APC-conjugated anti-T (TCRαβ) or anti-B (B220) cell mAb, and the percentages of injected T and B cells were analyzed by flow cytometry. Shown are percentages (mean±SDs) of injected WT (•) and CD47 KO (∘) T (FIG. 2C) and B (FIG. 2D) cells, which were normalized with the levels at 2 hour after cell transfer as 100%.

FIGS. 3A-3B depict the results of experiments in which clearance of porcine RBCs in CD47 KO animals was compared to WT mouse recipients. CFSE-stained pig RBCs (2×10⁸) were intravenously injected into CD47 KO (n=5) or WT (n=5) mice. FIG. 3A, top panels, contains FACS profiles showing percentages of porcine RBCs in the blood at the indicated times. Numbers indicate the percentages of CFSE+ porcine RBCs. FIG. 3A, bottom, is a graph depicting percentages (Mean±SDs) of porcine RBCs in blood, which were normalized with the levels at 15 min after injection as 100%. Results from 2 experiments are combined. * p<0.01; ** p<0.001. FIG. 3B is a set of photographs of spleen sections from CD47 KO (top row, ×100) and WT (middle row, ×100; bottom row, ×400) at 1 hour post injection of CFSE-stained pig RBCs, and frozen spleen sections were stained with anti-F4/80 mAb. Engulfment was seen as a yellow event after merging the green-filtered and red-filtered images (right column). Three mouse recipients from each group were examined and representative results are shown.

FIGS. 4A-4B are graphs depicting results of experiments in which spleen cells prepared from 12 week-old WT (n=3) and CD47 KO (n=3) mice were stained with R-phycoerythrin (R-PE) conjugated anti-mouse F4/80 (Caltag Laboratories, Burlingame, Calif.), and the percentages of F4/80+ macrophages were determined by flow cytometric analysis. FIG. 4A shows percentages (mean±SDs) of F4/80+ cells in the spleen. FIG. 4B shows numbers (mean±SDs) of F4/80+ cells per spleen.

FIGS. 5A-5C depict the results of experiments in which the expression of mouse CD47 on porcine cells and susceptibility of the cells to cytotoxicity by mouse macrophages. FIG. 5A, left panels, contains FACS profiles of expression of murine CD47 (mCD47) on transfected LCL-13271 pig tumor cell lines. Thin and bold histograms represent staining with isotype control and anti-mouse CD47 mAb (miap301), respectively. Neo transfectant LCL cells (LCL-neo), a representative clone (#1007) of mCD47 transfectant LCL cells (LCL-mCD47), and mouse CD47^{+/+} A20 cells are shown. FIG. 5A, right panels, contain photographs depicting the results of mCD47 RT-PCR. Lane 1, LCL-mCD47 cells (clone #1007); Lane 2, LCL-neo cells; Lane 3, non-transfected LCL-13271 cells; Lane 4, CD47^{+/+} mouse cell line A20. GAPDH was used as a DNA loading control. For FIG. 5B, LCL-mCD47 and LCL-neo cells were stained with different colors (CFSE or PKH-26), mixed at a 1:1 ratio, and cultured in culture plate (2.5×10⁴/well) with (◯) or without () WT mouse intraperitoneal macrophages (5×10⁵/well) for 3 days. FIG. 5B, left, is a graph showing are ratios of viable LCL-mCD47 to LCL-neo cells. FIG. 5B, right, is representative flow cytometric profiles (right; the percentages of LCL-mCD47 and LCL-neo cells are indicated) at the indicated time points. Combined results (Mean±SDs) from three independent experiments are presented. * p<0.05; ** p<0.01; *** p<0.001. FIG. 5C is a graph showing numbers of LCL-mCD47 (▪/) and LCL-neo (□/◯) cells in the upper transwell chambers (inside the transwells) in cultures, in which the lower chambers (outside transwells) contained either both target cells (i.e., a 1:1 mixture of LCL-mCD47 and LCL-neo cells) and mouse macrophages (T+M) or target cells only (T). Results (mean±SDs) from a representative experiment of three are shown.

FIGS. 6A-6B depict results of experiments which show that mouse CD47 expression attenuates phagocytosis of porcine cells in vitro by mouse macrophages. CFSE-labeled LCL-mCD47 or LCL-neo cells (2.5×10⁴/well) were incubated with mouse intraperitoneal macrophages (5×10⁵/well) in 96-well plate at 37° C. or 4° C. (controls); cultures were harvested 3 hours later and phagocytosis was determined by flow cytometry. FIG. 6A, left panel, depicts percent engulfment in Mac-1+ cells (mean±SDs of four experiments). FIG. 6A, right panel, depicts representative staining profiles showing engulfment (at 37° C., top) or background (4° C., bottom). FIG. 6B contains photographs of LCL-mCD47 and LCL-neo cells labeled with different colors (CFSE or PKH-26) were mixed at 1:1 ratio (2.5×10⁴ each) and cultured with 5×10⁵ CMAC-labeled mouse intraperitoneal macrophages for 3 hours, then non-engulfed target cells were washed off and phagocytosis was assessed by fluorescence microscopy. Pictures shown are images taken from an experiment, in which LCL-mCD47 and LCL-neo cells were labeled with CFSE and PKH-26, respectively. Data are representative of three experiments.

FIGS. 7A-7B are photographs depicting results of experiments which show that mouse CD47 expression attenuates in vivo phagocytosis of porcine cells. FIG. 7A. shows LCL-mCD47 and LCL-neo cells were labeled with CFSE and injected i.v. (1×10⁷/mouse) into C57BL/6 mice. At 3 hours after cell injection, spleens were harvested and stained with PE-conjugated anti-mouse F4/80 mAb. Engulfment was seen as a yellow event after merging the green-filtered and red-filtered images (right column). FIG. 7B shows cells from mice were injected i.v. with a 1:1 mixture of LCL-mCD47 and LCL-neo cells which were labeled with different colors (total 1×10⁷ cells per mouse). Spleens were harvested at 3 and 6 hours after cell injection. Pictures shown are representative images taken from an experiment, in which LCL-neo and LCL-mCD47 cells were labeled with PKH-26 and CFSE, respectively. Data were representative of three or more experiments.

Like reference symbols in the various drawings indicate like elements.

### DETAILED DESCRIPTION

Innate immune responses mediated by monocyte/macrophage cells shape the process of adaptive immunity. Phagocytic macrophages provide a first line of defense against invading microbes, and in turn present microbial antigens to T cells. Macrophages also internalize and present other types of nonself antigens, such as xenogeneic antigens, which can exacerbate immunological rejection of xenotransplants. Specific elimination of phagocytotic activity toward transplanted (e.g., xenogeneic) cells may attenuate subsequent T cell immune responses against xenogeneic antigens, while maintaining normal responses against pathogens. This facet of the immune response may be altered by genetically manipulating the xenogeneic cells to express, or increase expression, of an immune-inhibitory molecule that inhibits phagocytic activity. Alternatively, or in addition, immune responses may be altered with agents that bind and activate inhibitory signaling molecules on phagocytic cells.

Immune-Inhibitory Molecules

CD47 (also known as integrin-associated protein, or IAP) is a ubiquitously expressed 50 kDa transmembrane glycoprotein and is a member of the immunoglobulin superfamily. CD47 has a single extracellular IgV domain, a 5-TM1 region known as the multiple membrane-spanning (MMS) domain, and a short cytoplasmic tail that is alternatively spliced (Brown, Curr. Opin. Cell. Biol., 14(5):603-7, 2002; Brown and Frazier, Trends Cell Biol., 11(3):130-5, 2001). Amino acid sequences of human CD47 are found in GenBank® under the following accession numbers: NP_{—}001768.1 GI:4502673; NP 942088.1 GI:38683836; and NP_{—}001020250.1 GI:68223315. Nucleic acid sequences encoding human CD47 are found in GenBank® under the following accession numbers: NM_{—}001777.3 GI:68223312; NM_{—}198793.2 GI:68223313; and NM_{—}001025079.1 GI:68223314. Sequences of CD47 in other species are also known. See, for example, the amino acid sequences under the following accession numbers: XP_{—}516636.1 GI:55620774 (chimpanzee); XP_{—}535729.2 GI:74002601 (dog); NP_{—}034711.1 GI:6754382 (mouse); NP_{—}062068.1 GI:9506469 (rat); and XP_{—}416623.1 GI:50729702 (chicken).

Exemplary human CD47 amino acid and nucleic acid sequences are shown in Tables 1 and 2, respectively. The signal peptide of human CD47 corresponds to amino acids 1-18 of SEQ ID NO:1 (see SEQ ID NO:1 below, in Table 1). The extracellular domain of human CD47 corresponds to amino acids 1-142 of SEQ ID NO:1 (Motegi et al., EMBO J., 22(11): 2634-2644, 2003).

TABLE 1
Exemplary Human CD47 Amino Acid Sequences

| GenBank® GI and Acc. | |
|---|---|
| No. Isoform | Amino Acid Sequence |
| gi\|4502673\|ref\|NP_001768.1 | MWPLVAALLLGSACCGSAQLLFNKTKSVEFTFCNDTVVIPC |
| CD47 antigen isoform 1 | FVTNMEAQNTTEVYVKWKFKGRDIYTFDGALNKSTVPTDFS |
| precursor [Homo sapiens] | SAKIEVSQLLKGDASLKMDKSDAVSHTGNYTCEVTELTREG |
| | ETIIELKYRVVSWFSPNENILIVIFPIFAILLFWGQFGIKT |
| | LKYRSGGMDEKTIALLVAGLVITVIVIVGAILFVPGEYSLK |
| | NATGLGLIVTSTGILILLHYYVFSTAIGLTSFVIAILVIQV |
| | IAYILAVVGLSLCIAACIPMHGPLLISGLSILALAQLLGLV |
| | YMKFVASNQKTIQPPRKAVEEPLNAFKESKGMMNDE |
| | (SEQ ID NO: 1) |
| gi\|38683836\|ref\|NP_942088.1 | MWPLVAALLLGSACCGSAQLLFNKTKSVEFTFCNDTVVIPC |
| CD47 antigen isoform 2 | FVTNMEAQNTTEVYVKWKFKGRDIYTFDGALNKSTVPTDFS |
| precursor [Homo sapiens] | SAKIEVSQLLKGDASLKMDKSDAVSHTGNYTCEVTELTREG |
| | ETIIELKYRVVSWFSPNENILIVIFPIFAILLFWGQFGIKT |
| | LKYRSGGMDEKTIALLVAGLVITVIVIVGAILFVPGEYSLK |
| | NATGLGLIVTSTGILILLHYYVFSTAIGLTSFVIAILVIQV |
| | IAYILAVVGLSLCIAACIPMHGPLLISGLSILALAQLLGLV |
| | YMKFVASNQKTIQPPRNN (SEQ ID NO: 2) |
| gi\|68223315\|ref\|NP_001020250.1 | MWPLVAALLLGSACCGSAQLLFNKTKSVEFTFCNDTVVIPC |
| CD47 antigen isoform 3 | FVTNMEAQNTTEVYVKWKFKGRDIYTFDGALNKSTVPTDFS |
| precursor [Homo sapiens] | SAKIEVSQLLKGDASLKMDKSDAVSHTGNYTCEVTELTREG |
| | ETIIELKYRVVSWFSPNENILIVIFPIFAILLFWGQFGIKT |
| | LKYRSGGMDEKTIALLVAGLVITVIVIVGAILFVPGEYSLK |
| | NATGLGLIVTSTGILILLHYYVFSTAIGLTSFVIAILVIQV |
| | IAYILAVVGLSLCIAACIPMHGPLLISGLSILALAQLLGLV |
| | YMKFVASNQKTIQPPRKAVEEPLNE (SEQ ID NO: 3) |

TABLE 2
Exemplary Human CD47 Nucleic Acid Sequences

| | |
|---|---|
| gi\|68223312 | GGGGAGCAGGCGGGGGAGCGGGCGGGAAGCAGTGGGAGCGCGCGTGCGCGCGGCCGT |
| ref\|NM_001777.3 | GCAGCCTGGGCAGTGGGTCCTGCCTGTGACGCGCGGCGGCGGTCGGTCCTGCCTGTA |
| Homo sapiens | ACGGCGGCGGCGGCTGCTGCTCCAGACACCTGCGGCGGCGGCGGCGACCCCGCGGCG |
| CD47 molecule | GGCGCGGAGATGTGGCCCCTGGTAGCGGCGCTGTTGCTGGGCTCGGCGTGCTGCGGA |
| (CD47), | TCAGCTCAGCTACTATTTAATAAAACAAAATCTGTAGAATTCACGTTTTGTAATGAC |
| transcript | ACTGTCGTCATTCCATGCTTTGTTACTAATATGGAGGCACAAAACACTACTGAAGTA |
| variant 1, mRNA | TACGTAAAGTGGAAATTTAAAGGAAGAGATATTTACACCTTTGATGGAGCTCTAAAC |
| | AAGTCCACTGTCCCCACTGACTTTAGTAGTGCAAAAATTGAAGTCTCACAATTACTA |
| | AAAGGAGATGCCTCTTTGAAGATGGATAAGAGTGATGCTGTCTCACACACAGGAAAC |
| | TACACTTGTGAAGTAACAGAATTAACCAGAGAAGGTGAAACGATCATCGAGCTAAAA |
| | TATCGTGTTGTTTCATGGTTTTCTCCAAATGAAAATATTCTTATTGTTATTTTCCCA |
| | ATTTTTGCTATACTCCTGTTCTGGGGACAGTTTGGTATTAAAACACTTAAATATAGA |
| | TCCGGTGGTATGGATGAGAAAACAATTGCTTTACTTGTTGCTGGACTAGTGATCACT |
| | GTCATTGTCATTGTTGGAGCCATTCTTTTCGTCCCAGGTGAATATTCATTAAAGAAT |
| | GCTACTGGCCTTGGTTTAATTGTGACTTCTACAGGGATATTAATATTACTTCACTAC |
| | TATGTGTTTAGTACAGCGATTGGATTAACCTCCTTCGTCATTGCCATATTGGTTATT |
| | CAGGTGATAGCCTATATCCTCGCTGTGGTTGGACTGAGTCTCTGTATTGCGGCGTGT |
| | ATACCAATGCATGGCCCTCTTCTGATTTCAGGTTTGAGTATCTTAGCTCTAGCACAA |
| | TTACTTGGACTAGTTTATATGAAATTTGTGGCTTCCAATCAGAAGACTATACAACCT |
| | CCTAGGAAAGCTGTAGAGGAACCCCTTAATGCATTCAAAGAATCAAAAGGAATGATG |
| | AATGATGAATAACTGAAGTGAAGTGATGGACTCCGATTTGGAGAGTAGTAAGACGTG |
| | AAAGGAATACACTTGTGTTTAAGCACCATGGCCTTGATGATTCACTGTTGGGGAGAA |
| | GAAACAAGAAAAGTAACTGGTTGTCACCTATGAGACCCTTACGTGATTGTTAGTTAA |
| | GTTTTTATTCAAAGCAGCTGTAATTTAGTTAATAAAATAATTATGATCTATGTTGTT |
| | TGCCCAATTGAGATCCAGTTTTTTGTTGTTATTTTTAATCAATTAGGGGCAATAGTA |
| | GAATGGACAATTTCCAAGAATGATGCCTTTCAGGTCCTAGGGCCTCTGGCCTCTAGG |
| | TAACCAGTTTAAATTGGTTCAGGGTGATAACTACTTAGCACTGCCCTGGTGATTACC |
| | CAGAGATATCTATGAAAACCAGTGGCTTCCATCAAACCTTTGCCAACTCAGGTTCAC |
| | AGCAGCTTTGGGCAGTTATGGCAGTATGGCATTAGCTGAGAGGTGTCTGCCACTTCT |
| | GGGTCAATGGAATAATAAATTAAGTACAGGCAGGAATTTGGTTGGGAGCATCTTGTA |
| | TGATCTCCGTATGATGTGATATTGATGGAGATAGTGGTCCTCATTCTTGGGGGTTGC |
| | CATTCCCACATTCCCCCTTCAACAAACAGTGTAACAGGTCCTTCCCAGATTTAGGGT |
| | ACTTTTATTGATGGATATGTTTTCCTTTTATTCACATAACCCCTTGAAACCCTGTCT |
| | TGTCCTCCTGTTACTTGCTTCTGCTGTACAAGATGTAGCACCTTTTCTCCTCTTTGA |
| | ACATGGTCTAGTGACACGGTAGCACCAGTTGCAGGAAGGAGCCAGACTTGTTCTCAG |
| | AGCACTGTGTTCACACTTTTCAGCAAAAATAGCTATGGTTGTAACATATGTATTCCC |
| | TTCCTCTGATTTGAAGGCAAAAATCTACAGTGTTTCTTCACTTCTTTTCTGATCTGG |
| | GGCATGAAAAAAGCAAGATTGAAATTTGAACTATGAGTCTCCTGCATGGCAACAAAA |
| | TGTGTGTCACCATCAGGCCAACAGGCCAGCCCTTGAATGGGGATTTATTACTGTTGT |
| | ATCTATGTTGCATGATAAACATTCATCACCTTCCTCCTGTAGTCCTGCCTCGTACTC |
| | CCCTTCCCCTATGATTGAAAAGTAAACAAAACCCACATTTCCTATCCTGGTTAGAAG |
| | AAAATTAATGTTCTGACAGTTGTGATCGCCTGGAGTACTTTTAGACTTTTAGCATTC |
| | GTTTTTTACCTGTTTGTGGATGTGTGTTTGTATGTGCATACGTATGAGATAGGCACA |
| | TGCATCTTCTGTATGGACAAAGGTGGGGTACCTACAGGAGAGCAAAGGTTAATTTTG |
| | TGCTTTTAGTAAAAACATTTAAATACAAAGTTCTTTATTGGGTGGAATTATATTTGA |
| | TGCAAATATTTGATCACTTAAAACTTTTAAAACTTCTAGGTAATTTGCCACGCTTTT |
| | TGACTGCTCACCAATACCCTGTAAAAATACGTAATTCTTCCTGTTTGTGTAATAAGA |
| | TATTCATATTTGTAGTTGCATTAATAATAGTTATTTCTTAGTCCATCAGATGTTCCC |
| | GTGTGCCTCTTTTATGCCAAATTGATTGTCATATTTCATGTTGGGACCAAGTAGTTT |
| | GCCCATGGCAAACCTAAATTTATGACCTGCTGAGGCCTCTCAGAAAACTGAGCATAC |
| | TAGCAAGACAGCTCTTCTTGAAAAAAAAAATATGTATACACAAATATATACGTATAT |
| | CTATATATACGTATGTATATACACACATGTATATTCTTCCTTGATTGTGTAGCTGTC |
| | CAAAATAATAACATATATAGAGGGAGCTGTATTCCTTTATACAAATCTGATGGCTCC |
| | TGCAGCACTTTTTCCTTCTGAAAATATTTACATTTTGCTAACCTAGTTTGTTACTTT |
| | AAAAATCAGTTTTGATGAAAGGAGGGAAAAGCAGATGGACTTGAAAAAGATCCAAGC |
| | TCCTATTAGAAAAGGTATGAAAATCTTTATAGTAAAATTTTTTATAAACTAAAGTTG |
| | TACCTTTTAATATGTAGTAAACTCTCATTTATTTGGGGTTCGCTCTTGGATCTCATC |
| | CATCCATTGTGTTCTCTTTAATGCTGCCTGCCTTTTGAGGCATTCACTGCCCTAGAC |
| | AATGCCACCAGAGATAGTGGGGGAAATGCCAGATGAAACCAACTCTTGCTCTCACTA |
| | GTTGTCAGCTTCTCTGGATAAGTGACCACAGAAGCAGGAGTCCTCCTGCTTGGGCAT |
| | CATTGGGCCAGTTCCTTCTCTTTAAATCAGATTTGTAATGGCTCCCAAATTCCATCA |
| | CATCACATTTAAATTGCAGACAGTGTTTTGCACATCATGTATCTGTTTTGTCCCATA |
| | ATATGCTTTTTACTCCCTGATCCCAGTTTCTGCTGTTGACTCTTCCATTCAGTTTTA |
| | TTTATTGTGTGTTCTCACAGTGACACCATTTGTCCTTTTCTGCAACAACCTTTCCAG |
| | CTACTTTTGCCAAATTCTATTTGTCTTCTCCTTCAAAACATTCTCCTTTGCAGTTCC |
| | TCTTCATCTGTGTAGCTGCTCTTTTGTCTCTTAACTTACCATTCCTATAGTACTTTA |
| | TGCATCTCTGCTTAGTTCTATTAGTTTTTTGGCCTTGCTCTTCTCCTTGATTTTAAA |
| | ATTCCTTCTATAGCTAGAGCTTTTCTTTCTTTCATTCTCTCTTCCTGCAGTGTTTTG |
| | CATACATCAGAAGCTAGGTACATAAGTTAAATGATTGAGAGTTGGCTGTATTTAGAT |
| | TTATCACTTTTTAATAGGGTGAGCTTGAGAGTTTTCTTTCTTTCTGTTTTTTTTTTT |
| | TGTTTTTTTTTTTTTTTTTTTTTTTTTTTTTTTTGACTAATTTCACATGCTCTAAAA |
| | ACCTTCAAAGGTGATTATTTTTCTCCTGGAAACTCCAGGTCCATTCTGTTTAAATCC |
| | CTAAGAATGTCAGAATTAAAATAACAGGGCTATCCCGTAATTGGAAATATTTCTTTT |
| | TTCAGGATGCTATAGTCAATTTAGTAAGTGACCACCAAATTGTTATTTGCACTAACA |
| | AAGCTCAAAACACGATAAGTTTACTCCTCCATCTCAGTAATAAAAATTAAGCTGTAA |
| | TCAACCTTCTAGGTTTCTCTTGTCTTAAAATGGGTATTCAAAAATGGGGATCTGTGG |
| | TGTATGTATGGAAACACATACTCCTTAATTTACCTGTTGTTGGAAACTGGAGAAATG |
| | ATTGTCGGGCAACCGTTTATTTTTTATTGTATTTTATTTGGTTGAGGGATTTTTTTA |
| | TAAACAGTTTTACTTGTGTCATATTTTAAAATTACTAACTGCCATCACCTGCTGGGG |
| | TCCTTTGTTAGGTCATTTTCAGTGACTAATAGGGATAATCCAGGTAACTTTGAAGAG |
| | ATGAGCAGTGAGTGACCAGGCAGTTTTTCTGCCTTTAGCTTTGACAGTTCTTAATTA |
| | AGATCATTGAAGACCAGCTTTCTCATAAATTTCTCTTTTTGAAAAAAAGAAAGCATT |
| | TGTACTAAGCTCCTCTGTAAGACAACATCTTAAATCTTAAAAGTGTTGTTATCATGA |
| | CTGGTGAGAGAAGAAAACATTTTGTTTTTATTAAATGGAGCATTATTTACAAAAAGC |
| | CATTGTTGAGAATTAGATCCCACATCGTATAAATATCTATTAACCATTCTAAATAAA |
| | GAGAACTCCAGTGTTGCTATGTGCAAGATCCTCTCTTGGAGCTTTTTTGCATAGCAA |
| | TTAAAGGTGTGCTATTTGTCAGTAGCCATTTTTTTGCAGTGATTTGAAGACCAAAGT |
| | TGTTTTACAGCTGTGTTACCGTTAAAGGTTTTTTTTTTTATATGTATTAAATCAATT |
| | TATCACTGTTTAAAGCTTTGAATATCTGCAATCTTTGCCAAGGTACTTTTTTATTTA |
| | AAAAAAAACATAACTTTGTAAATATTACCCTGTAATATTATATATACTTAATAAAAC |
| | ATTTTAAGCTATTTTGTTGGGCTATTTCTATTGCTGCTACAGCAGACCACAAGCACA |
| | TTTCTGAAAAATTTAATTTATTAATGTATTTTTAAGTTGCTTATATTCTAGGTAACA |
| | ATGTAAAGAATGATTTAAAATATTAATTATGAATTTTTTGAGTATAATACCCAATAA |
| | GCTTTTAATTAGAGCAGAGTTTTAATTAAAAGTTTTAAATCAGTC |
| | (SEQ ID NO: 4) |
| gi\|68223313\|ref\| | GGGGAGCAGGCGGGGGAGCGGGCGGGAAGCAGTGGGAGCGCGCGTGCGCGCGGCCGT |
| NM_198793.2\| | GCAGCCTGGGCAGTGGGTCCTGCCTGTGACGCGCGGCGGCGGTCGGTCCTGCCTGTA |
| Homo sapiens | ACGGCGGCGGCGGCTGCTGCTCCAGACACCTGCGGCGGCGGCGGCGACCCCGCGGCG |
| CD47 molecule | GGCGCGGAGATGTGGCCCCTGGTAGCGGCGCTGTTGCTGGGCTCGGCGTGCTGCGGA |
| (CD47), | TCAGCTCAGCTACTATTTAATAAAACAAAATCTGTAGAATTCACGTTTTGTAATGAC |
| transcript | ACTGTCGTCATTCCATGCTTTGTTACTAATATGGAGGCACAAAACACTACTGAAGTA |
| variant 2, mRNA | TACGTAAAGTGGAAATTTAAAGGAAGAGATATTTACACCTTTGATGGAGCTCTAAAC |
| | AAGTCCACTGTCCCCACTGACTTTAGTAGTGCAAAAATTGAAGTCTCACAATTACTA |
| | AAAGGAGATGCCTCTTTGAAGATGGATAAGAGTGATGCTGTCTCACACACAGGAAAC |
| | TACACTTGTGAAGTAACAGAATTAACCAGAGAAGGTGAAACGATCATCGAGCTAAAA |
| | TATCGTGTTGTTTCATGGTTTTCTCCAAATGAAAATATTCTTATTGTTATTTTCCCA |
| | ATTTTTGCTATACTCCTGTTCTGGGGACAGTTTGGTATTAAAACACTTAAATATAGA |
| | TCCGGTGGTATGGATGAGAAAACAATTGCTTTACTTGTTGCTGGACTAGTGATCACT |
| | GTCATTGTCATTGTTGGAGCCATTCTTTTCGTCCCAGGTGAATATTCATTAAAGAAT |
| | GCTACTGGCCTTGGTTTAATTGTGACTTCTACAGGGATATTAATATTACTTCACTAC |
| | TATGTGTTTAGTACAGCGATTGGATTAACCTCCTTCGTCATTGCCATATTGGTTATT |
| | CAGGTGATAGCCTATATCCTCGCTGTGGTTGGACTGAGTCTCTGTATTGCGGCGTGT |
| | ATACCAATGCATGGCCCTCTTCTGATTTCAGGTTTGAGTATCTTAGCTCTAGCACAA |
| | TTACTTGGACTAGTTTATATGAAATTTGTGGCTTCCAATCAGAAGACTATACAACCT |
| | CCTAGGAATAACTGAAGTGAAGTGATGGACTCCGATTTGGAGAGTAGTAAGACGTGA |
| | AAGGAATACACTTGTGTTTAAGCACCATGGCCTTGATGATTCACTGTTGGGGAGAAG |
| | AAACAAGAAAAGTAACTGGTTGTCACCTATGAGACCCTTACGTGATTGTTAGTTAAG |
| | TTTTTATTCAAAGCAGCTGTAATTTAGTTAATAAAATAATTATGATCTATGTTGTTT |
| | GCCCAATTGAGATCCAGTTTTTTGTTGTTATTTTTAATCAATTAGGGGCAATAGTAG |
| | AATGGACAATTTCCAAGAATGATGCCTTTCAGGTCCTAGGGCCTCTGGCCTCTAGGT |
| | AACCAGTTTAAATTGGTTCAGGGTGATAACTACTTAGCACTGCCCTGGTGATTACCC |
| | AGAGATATCTATGAAAACCAGTGGCTTCCATCAAACCTTTGCCAACTCAGGTTCACA |
| | GCAGCTTTGGGCAGTTATGGCAGTATGGCATTAGCTGAGAGGTGTCTGCCACTTCTG |
| | GGTCAATGGAATAATAAATTAAGTACAGGCAGGAATTTGGTTGGGAGCATCTTGTAT |
| | GATCTCCGTATGATGTGATATTGATGGAGATAGTGGTCCTCATTCTTGGGGGTTGCC |
| | ATTCCCACATTCCCCCTTCAACAAACAGTGTAACAGGTCCTTCCCAGATTTAGGGTA |
| | CTTTTATTGATGGATATGTTTTCCTTTTATTCACATAACCCCTTGAAACCCTGTCTT |
| | GTCCTCCTGTTACTTGCTTCTGCTGTACAAGATGTAGCACCTTTTCTCCTCTTTGAA |
| | CATGGTCTAGTGACACGGTAGCACCAGTTGCAGGAAGGAGCCAGACTTGTTCTCAGA |
| | GCACTGTGTTCACACTTTTCAGCAAAAATAGCTATGGTTGTAACATATGTATTCCCT |
| | TCCTCTGATTTGAAGGCAAAAATCTACAGTGTTTCTTCACTTCTTTTCTGATCTGGG |
| | GCATGAAAAAAGCAAGATTGAAATTTGAACTATGAGTCTCCTGCATGGCAACAAAAT |
| | GTGTGTCACCATCAGGCCAACAGGCCAGCCCTTGAATGGGGATTTATTACTGTTGTA |
| | TCTATGTTGCATGATAAACATTCATCACCTTCCTCCTGTAGTCCTGCCTCGTACTCC |
| | CCTTCCCCTATGATTGAAAAGTAAACAAAACCCACATTTCCTATCCTGGTTAGAAGA |
| | AAATTAATGTTCTGACAGTTGTGATCGCCTGGAGTACTTTTAGACTTTTAGCATTCG |
| | TTTTTTACCTGTTTGTGGATGTGTGTTTGTATGTGCATACGTATGAGATAGGCACAT |
| | GCATCTTCTGTATGGACAAAGGTGGGGTACCTACAGGAGAGCAAAGGTTAATTTTGT |
| | GCTTTTAGTAAAAACATTTAAATACAAAGTTCTTTATTGGGTGGAATTATATTTGAT |
| | GCAAATATTTGATCACTTAAAACTTTTAAAACTTCTAGGTAATTTGCCACGCTTTTT |
| | GACTGCTCACCAATACCCTGTAAAAATACGTAATTCTTCCTGTTTGTGTAATAAGAT |
| | ATTCATATTTGTAGTTGCATTAATAATAGTTATTTCTTAGTCCATCAGATGTTCCCG |
| | TGTGCCTCTTTTATGCCAAATTGATTGTCATATTTCATGTTGGGACCAAGTAGTTTG |
| | CCCATGGCAAACCTAAATTTATGACCTGCTGAGGCCTCTCAGAAAACTGAGCATACT |
| | AGCAAGACAGCTCTTCTTGAAAAAAAAAATATGTATACACAAATATATACGTATATC |
| | TATATATACGTATGTATATACACACATGTATATTCTTCCTTGATTGTGTAGCTGTCC |
| | AAAATAATAACATATATAGAGGGAGCTGTATTCCTTTATACAAATCTGATGGCTCCT |
| | GCAGCACTTTTTCCTTCTGAAAATATTTACATTTTGCTAACCTAGTTTGTTACTTTA |
| | AAAATCAGTTTTGATGAAAGGAGGGAAAAGCAGATGGACTTGAAAAAGATCCAAGCT |
| | CCTATTAGAAAAGGTATGAAAATCTTTATAGTAAAATTTTTTATAAACTAAAGTTGT |
| | ACCTTTTAATATGTAGTAAACTCTCATTTATTTGGGGTTCGCTCTTGGATCTCATCC |
| | ATCCATTGTGTTCTCTTTAATGCTGCCTGCCTTTTGAGGCATTCACTGCCCTAGACA |
| | ATGCCACCAGAGATAGTGGGGGAAATGCCAGATGAAACCAACTCTTGCTCTCACTAG |
| | TTGTCAGCTTCTCTGGATAAGTGACCACAGAAGCAGGAGTCCTCCTGCTTGGGCATC |
| | ATTGGGCCAGTTCCTTCTCTTTAAATCAGATTTGTAATGGCTCCCAAATTCCATCAC |
| | ATCACATTTAAATTGCAGACAGTGTTTTGCACATCATGTATCTGTTTTGTCCCATAA |
| | TATGCTTTTTACTCCCTGATCCCAGTTTCTGCTGTTGACTCTTCCATTCAGTTTTAT |
| | TTATTGTGTGTTCTCACAGTGACACCATTTGTCCTTTTCTGCAACAACCTTTCCAGC |
| | TACTTTTGCCAAATTCTATTTGTCTTCTCCTTCAAAACATTCTCCTTTGCAGTTCCT |
| | CTTCATCTGTGTAGCTGCTCTTTTGTCTCTTAACTTACCATTCCTATAGTACTTTAT |
| | GCATCTCTGCTTAGTTCTATTAGTTTTTTGGCCTTGCTCTTCTCCTTGATTTTAAAA |
| | TTCCTTCTATAGCTAGAGCTTTTCTTTCTTTCATTCTCTCTTCCTGCAGTGTTTTGC |
| | ATACATCAGAAGCTAGGTACATAAGTTAAATGATTGAGAGTTGGCTGTATTTAGATT |
| | TATCACTTTTTAATAGGGTGAGCTTGAGAGTTTTCTTTCTTTCTGTTTTTTTTTTTT |
| | GTTTTTTTTTTTTTTTTTTTTTTTTTTTTTTTTGACTAATTTCACATGCTCTAAAAA |
| | CCTTCAAAGGTGATTATTTTTCTCCTGGAAACTCCAGGTCCATTCTGTTTAAATCCC |
| | TAAGAATGTCAGAATTAAAATAACAGGGCTATCCCGTAATTGGAAATATTTCTTTTT |
| | TCAGGATGCTATAGTCAATTTAGTAAGTGACCACCAAATTGTTATTTGCACTAACAA |
| | AGCTCAAAACACGATAAGTTTACTCCTCCATCTCAGTAATAAAAATTAAGCTGTAAT |
| | CAACCTTCTAGGTTTCTCTTGTCTTAAAATGGGTATTCAAAAATGGGGATCTGTGGT |
| | GTATGTATGGAAACACATACTCCTTAATTTACCTGTTGTTGGAAACTGGAGAAATGA |
| | TTGTCGGGCAACCGTTTATTTTTTATTGTATTTTATTTGGTTGAGGGATTTTTTTAT |
| | AAACAGTTTTACTTGTGTCATATTTTAAAATTACTAACTGCCATCACCTGCTGGGGT |
| | CCTTTGTTAGGTCATTTTCAGTGACTAATAGGGATAATCCAGGTAACTTTGAAGAGA |
| | TGAGCAGTGAGTGACCAGGCAGTTTTTCTGCCTTTAGCTTTGACAGTTCTTAATTAA |
| | GATCATTGAAGACCAGCTTTCTCATAAATTTCTCTTTTTGAAAAAAAGAAAGCATTT |
| | GTACTAAGCTCCTCTGTAAGACAACATCTTAAATCTTAAAAGTGTTGTTATCATGAC |
| | TGGTGAGAGAAGAAAACATTTTGTTTTTATTAAATGGAGCATTATTTACAAAAAGCC |
| | ATTGTTGAGAATTAGATCCCACATCGTATAAATATCTATTAACCATTCTAAATAAAG |
| | AGAACTCCAGTGTTGCTATGTGCAAGATCCTCTCTTGGAGCTTTTTTGCATAGCAAT |
| | TAAAGGTGTGCTATTTGTCAGTAGCCATTTTTTTGCAGTGATTTGAAGACCAAAGTT |
| | GTTTTACAGCTGTGTTACCGTTAAAGGTTTTTTTTTTTATATGTATTAAATCAATTT |
| | ATCACTGTTTAAAGCTTTGAATATCTGCAATCTTTGCCAAGGTACTTTTTTATTTAA |
| | AAAAAAACATAACTTTGTAAATATTACCCTGTAATATTATATATACTTAATAAAACA |
| | TTTTAAGCTATTTTGTTGGGCTATTTCTATTGCTGCTACAGCAGACCACAAGCACAT |
| | TTCTGAAAAATTTAATTTATTAATGTATTTTTAAGTTGCTTATATTCTAGGTAACAA |
| | TGTAAAGAATGATTTAAAATATTAATTATGAATTTTTTGAGTATAATACCCAATAAG |
| | CTTTTAATTAGAGCAGAGTTTTAATTAAAAGTTTTAAATCAGTC |
| | (SEQ ID NO: 5) |
| gi\|68223314\|ref\| | GGGGAGCAGGCGGGGGAGCGGGCGGGAAGCAGTGGGAGCGCGCGTGCGCGCGGCCGT |
| NM_001025079.1 | GCAGCCTGGGCAGTGGGTCCTGCCTGTGACGCGCGGCGGCGGTCGGTCCTGCCTGTA |
| Homo sapiens | ACGGCGGCGGCGGCTGCTGCTCCAGACACCTGCGGCGGCGGCGGCGACCCCGCGGCG |
| CD47 molecule | GGCGCGGAGATGTGGCCCCTGGTAGCGGCGCTGTTGCTGGGCTCGGCGTGCTGCGGA |
| (CD47), | TCAGCTCAGCTACTATTTAATAAAACAAAATCTGTAGAATTCACGTTTTGTAATGAC |
| transcript | ACTGTCGTCATTCCATGCTTTGTTACTAATATGGAGGCACAAAACACTACTGAAGTA |
| variant 3, mRNA | TACGTAAAGTGGAAATTTAAAGGAAGAGATATTTACACCTTTGATGGAGCTCTAAAC |
| | AAGTCCACTGTCCCCACTGACTTTAGTAGTGCAAAAATTGAAGTCTCACAATTACTA |
| | AAAGGAGATGCCTCTTTGAAGATGGATAAGAGTGATGCTGTCTCACACACAGGAAAC |
| | TACACTTGTGAAGTAACAGAATTAACCAGAGAAGGTGAAACGATCATCGAGCTAAAA |
| | TATCGTGTTGTTTCATGGTTTTCTCCAAATGAAAATATTCTTATTGTTATTTTCCCA |
| | ATTTTTGCTATACTCCTGTTCTGGGGACAGTTTGGTATTAAAACACTTAAATATAGA |
| | TCCGGTGGTATGGATGAGAAAACAATTGCTTTACTTGTTGCTGGACTAGTGATCACT |
| | GTCATTGTCATTGTTGGAGCCATTCTTTTCGTCCCAGGTGAATATTCATTAAAGAAT |
| | GCTACTGGCCTTGGTTTAATTGTGACTTCTACAGGGATATTAATATTACTTCACTAC |
| | TATGTGTTTAGTACAGCGATTGGATTAACCTCCTTCGTCATTGCCATATTGGTTATT |
| | CAGGTGATAGCCTATATCCTCGCTGTGGTTGGACTGAGTCTCTGTATTGCGGCGTGT |
| | ATACCAATGCATGGCCCTCTTCTGATTTCAGGTTTGAGTATCTTAGCTCTAGCACAA |
| | TTACTTGGACTAGTTTATATGAAATTTGTGGCTTCCAATCAGAAGACTATACAACCT |
| | CCTAGGAAAGCTGTAGAGGAACCCCTTAATGAATAACTGAAGTGAAGTGATGGACTC |
| | CGATTTGGAGAGTAGTAAGACGTGAAAGGAATACACTTGTGTTTAAGCACCATGGCC |
| | TTGATGATTCACTGTTGGGGAGAAGAAACAAGAAAAGTAACTGGTTGTCACCTATGA |
| | GACCCTTACGTGATTGTTAGTTAAGTTTTTATTCAAAGCAGCTGTAATTTAGTTAAT |
| | AAAATAATTATGATCTATGTTGTTTGCCCAATTGAGATCCAGTTTTTTGTTGTTATT |
| | TTTAATCAATTAGGGGCAATAGTAGAATGGACAATTTCCAAGAATGATGCCTTTCAG |
| | GTCCTAGGGCCTCTGGCCTCTAGGTAACCAGTTTAAATTGGTTCAGGGTGATAACTA |
| | CTTAGCACTGCCCTGGTGATTACCCAGAGATATCTATGAAAACCAGTGGCTTCCATC |
| | AAACCTTTGCCAACTCAGGTTCACAGCAGCTTTGGGCAGTTATGGCAGTATGGCATT |
| | AGCTGAGAGGTGTCTGCCACTTCTGGGTCAATGGAATAATAAATTAAGTACAGGCAG |
| | GAATTTGGTTGGGAGCATCTTGTATGATCTCCGTATGATGTGATATTGATGGAGATA |
| | GTGGTCCTCATTCTTGGGGGTTGCCATTCCCACATTCCCCCTTCAACAAACAGTGTA |
| | ACAGGTCCTTCCCAGATTTAGGGTACTTTTATTGATGGATATGTTTTCCTTTTATTC |
| | ACATAACCCCTTGAAACCCTGTCTTGTCCTCCTGTTACTTGCTTCTGCTGTACAAGA |
| | TGTAGCACCTTTTCTCCTCTTTGAACATGGTCTAGTGACACGGTAGCACCAGTTGCA |
| | GGAAGGAGCCAGACTTGTTCTCAGAGCACTGTGTTCACACTTTTCAGCAAAAATAGC |
| | TATGGTTGTAACATATGTATTCCCTTCCTCTGATTTGAAGGCAAAAATCTACAGTGT |
| | TTCTTCACTTCTTTTCTGATCTGGGGCATGAAAAAAGCAAGATTGAAATTTGAACTA |
| | TGAGTCTCCTGCATGGCAACAAAATGTGTGTCACCATCAGGCCAACAGGCCAGCCCT |
| | TGAATGGGGATTTATTACTGTTGTATCTATGTTGCATGATAAACATTCATCACCTTC |
| | CTCCTGTAGTCCTGCCTCGTACTCCCCTTCCCCTATGATTGAAAAGTAAACAAAACC |
| | CACATTTCCTATCCTGGTTAGAAGAAAATTAATGTTCTGACAGTTGTGATCGCCTGG |
| | AGTACTTTTAGACTTTTAGCATTCGTTTTTTACCTGTTTGTGGATGTGTGTTTGTAT |
| | GTGCATACGTATGAGATAGGCACATGCATCTTCTGTATGGACAAAGGTGGGGTACCT |
| | ACAGGAGAGCAAAGGTTAATTTTGTGCTTTTAGTAAAAACATTTAAATACAAAGTTC |
| | TTTATTGGGTGGAATTATATTTGATGCAAATATTTGATCACTTAAAACTTTTAAAAC |
| | TTCTAGGTAATTTGCCACGCTTTTTGACTGCTCACCAATACCCTGTAAAAATACGTA |
| | ATTCTTCCTGTTTGTGTAATAAGATATTCATATTTGTAGTTGCATTAATAATAGTTA |
| | TTTCTTAGTCCATCAGATGTTCCCGTGTGCCTCTTTTATGCCAAATTGATTGTCATA |
| | TTTCATGTTGGGACCAAGTAGTTTGCCCATGGCAAACCTAAATTTATGACCTGCTGA |
| | GGCCTCTCAGAAAACTGAGCATACTAGCAAGACAGCTCTTCTTGAAAAAAAAAATAT |
| | GTATACACAAATATATACGTATATCTATATATACGTATGTATATACACACATGTATA |
| | TTCTTCCTTGATTGTGTAGCTGTCCAAAATAATAACATATATAGAGGGAGCTGTATT |
| | CCTTTATACAAATCTGATGGCTCCTGCAGCACTTTTTCCTTCTGAAAATATTTACAT |
| | TTTGCTAACCTAGTTTGTTACTTTAAAAATCAGTTTTGATGAAAGGAGGGAAAAGCA |
| | GATGGACTTGAAAAAGATCCAAGCTCCTATTAGAAAAGGTATGAAAATCTTTATAGT |
| | AAAATTTTTTATAAACTAAAGTTGTACCTTTTAATATGTAGTAAACTCTCATTTATT |
| | TGGGGTTCGCTCTTGGATCTCATCCATCCATTGTGTTCTCTTTAATGCTGCCTGCCT |
| | TTTGAGGCATTCACTGCCCTAGACAATGCCACCAGAGATAGTGGGGGAAATGCCAGA |
| | TGAAACCAACTCTTGCTCTCACTAGTTGTCAGCTTCTCTGGATAAGTGACCACAGAA |
| | GCAGGAGTCCTCCTGCTTGGGCATCATTGGGCCAGTTCCTTCTCTTTAAATCAGATT |
| | TGTAATGGCTCCCAAATTCCATCACATCACATTTAAATTGCAGACAGTGTTTTGCAC |
| | ATCATGTATCTGTTTTGTCCCATAATATGCTTTTTACTCCCTGATCCCAGTTTCTGC |
| | TGTTGACTCTTCCATTCAGTTTTATTTATTGTGTGTTCTCACAGTGACACCATTTGT |
| | CCTTTTCTGCAACAACCTTTCCAGCTACTTTTGCCAAATTCTATTTGTCTTCTCCTT |
| | CAAAACATTCTCCTTTGCAGTTCCTCTTCATCTGTGTAGCTGCTCTTTTGTCTCTTA |
| | ACTTACCATTCCTATAGTACTTTATGCATCTCTGCTTAGTTCTATTAGTTTTTTGGC |
| | CTTGCTCTTCTCCTTGATTTTAAAATTCCTTCTATAGCTAGAGCTTTTCTTTCTTTC |
| | ATTCTCTCTTCCTGCAGTGTTTTGCATACATCAGAAGCTAGGTACATAAGTTAAATG |
| | ATTGAGAGTTGGCTGTATTTAGATTTATCACTTTTTAATAGGGTGAGCTTGAGAGTT |
| | TTCTTTCTTTCTGTTTTTTTTTTTTGTTTTTTTTTTTTTTTTTTTTTTTTTTTTTTT |
| | TGACTAATTTCACATGCTCTAAAAACCTTCAAAGGTGATTATTTTTCTCCTGGAAAC |
| | TCCAGGTCCATTCTGTTTAAATCCCTAAGAATGTCAGAATTAAAATAACAGGGCTAT |
| | CCCGTAATTGGAAATATTTCTTTTTTCAGGATGCTATAGTCAATTTAGTAAGTGACC |
| | ACCAAATTGTTATTTGCACTAACAAAGCTCAAAACACGATAAGTTTACTCCTCCATC |
| | TCAGTAATAAAAATTAAGCTGTAATCAACCTTCTAGGTTTCTCTTGTCTTAAAATGG |
| | GTATTCAAAAATGGGGATCTGTGGTGTATGTATGGAAACACATACTCCTTAATTTAC |
| | CTGTTGTTGGAAACTGGAGAAATGATTGTCGGGCAACCGTTTATTTTTTATTGTATT |
| | TTATTTGGTTGAGGGATTTTTTTATAAACAGTTTTACTTGTGTCATATTTTAAAATT |
| | ACTAACTGCCATCACCTGCTGGGGTCCTTTGTTAGGTCATTTTCAGTGACTAATAGG |
| | GATAATCCAGGTAACTTTGAAGAGATGAGCAGTGAGTGACCAGGCAGTTTTTCTGCC |
| | TTTAGCTTTGACAGTTCTTAATTAAGATCATTGAAGACCAGCTTTCTCATAAATTTC |
| | TCTTTTTGAAAAAAAGAAAGCATTTGTACTAAGCTCCTCTGTAAGACAACATCTTAA |
| | ATCTTAAAAGTGTTGTTATCATGACTGGTGAGAGAAGAAAACATTTTGTTTTTATTA |
| | AATGGAGCATTATTTACAAAAAGCCATTGTTGAGAATTAGATCCCACATCGTATAAA |
| | TATCTATTAACCATTCTAAATAAAGAGAACTCCAGTGTTGCTATGTGCAAGATCCTC |
| | TCTTGGAGCTTTTTTGCATAGCAATTAAAGGTGTGCTATTTGTCAGTAGCCATTTTT |
| | TTGCAGTGATTTGAAGACCAAAGTTGTTTTACAGCTGTGTTACCGTTAAAGGTTTTT |
| | TTTTTTATATGTATTAAATCAATTTATCACTGTTTAAAGCTTTGAATATCTGCAATC |
| | TTTGCCAAGGTACTTTTTTATTTAAAAAAAAACATAACTTTGTAAATATTACCCTGT |
| | AATATTATATATACTTAATAAAACATTTTAAGCTATTTTGTTGGGCTATTTCTATTG |
| | CTGCTACAGCAGACCACAAGCACATTTCTGAAAAATTTAATTTATTAATGTATTTTT |
| | AAGTTGCTTATATTCTAGGTAACAATGTAAAGAATGATTTAAAATATTAATTATGAA |
| | TTTTTTGAGTATAATACCCAATAAGCTTTTAATTAGAGCAGAGTTTTAATTAAAAGT |
| | TTTAAATCAGTC (SEQ ID NO: 6) |

Other immune-inhibitory molecules suitable for the methods and compositions described herein are those which interact inefficiently, or fail to interact, with counterpart ligands which is derived from another species (i.e., the ligands have low cross-reactivity across species barriers). Exemplary molecules include CD200, ligands for paired Ig-like receptor (PIR)-B, ligands for immunoglobulin-like transcript (ILT)3, and ligands for CD33-related receptors. CD200 is a type-1 membrane glycoprotein and is a member of the immunoglobulin (Ig) superfamily. Sequences for human CD200 are found under accession nos. NP_{—}005935.4 GI:90903247 and NP_{—}001004196.2 GI:90903245. ILT3 is also a member of the Ig superfamily. The cloning of a human ILT3 sequence is described in Cella et al., J. Exp. Med., 185(10):1743-1751, 1997. CD33-related receptors are discussed in Crocker and Varki, 1: Trends Immunol., 22(6):337-42, 2001.

In various embodiments, an immune-inhibitory molecule includes a sequence at least 80%, 85%, 90%, 95%, 96%, 97%, 98%, 99% identical to a wild-type sequence (e.g., a human CD47 amino sequence), or a fragment thereof (e.g., the molecule has a sequence at least 80%, 85%, 90%, 95%, 96%, 97%, 98%, or 99% identical to the human CD47 amino sequence of SEQ ID NO:1, or a fragment thereof). In various embodiments, the immune-inhibitory molecule has a sequence which differs from the sequence of a wild-type sequence in at least 1 amino acid position, but not more than 35 amino acid positions (e.g., the sequence differs from SEQ ID NO:1 at 1, 2, 3, 4, 5, 6, 7, 8, 9, 10, 11, 12, 13, 14, 15, 16, 17, 18, 19, 20, 21, 22, 23, 24, or 25 amino acid positions).

Useful fragments and variants include those which retain the ability to bind with the appropriate receptor on an immune cell (e.g., a fragment which binds to SIRPα on a macrophage) and mediate at least one biological activity of the molecule (e.g., inhibition of phagocytosis, stimulation of tyrosine phosphorylation of SIRPα). For example, a cell of a first species (e.g., swine) which expresses a polypeptide including the fragment or variant is less susceptible to phagocytosis by a phagocytic cell (e.g., a macrophage) of a second species, as compared to a control (e.g., a cell which does not express the fragment or variant). Polypeptides which include all or a portion of the extracellular domain of CD47 are contemplated. See, e.g., Motegi et al., EMBO J., 22(11): 2634-2644, 2003, which describes the construction of a human CD47-Fc fusion protein. The polypeptides may be fusion proteins and may be membrane-associated or soluble forms.

The practice of the present invention will employ, unless otherwise indicated, techniques which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

Genetically Engineered Cells

Transgenic cells (e.g., transgenic swine cells) can be produced by any methods known to those in the art. Transgenes can be introduced into cells, e.g., stem cells, e.g., cultured stem cells, by any methods which allows expression of these genes, e.g., at a level and for a period sufficient to inhibit an immunological reaction to the cell (e.g., a macrophage-mediated immune reaction), e.g., to promote engraftment or maintenance of the cells. These methods include e.g., transfection, electroporation, particle gun bombardment, and transduction by viral vectors, e.g., by retroviruses. Transgenic cells can also be derived from transgenic animals.

Retroviral Introduction of Transgenes

Recombinant retroviruses are useful vehicles for gene transfer, see e.g., Eglitis et al., 1988, Adv. Exp. Med. Biol. 241:19. In one example of a retroviral vector construct, the structural genes of the virus are replaced by a single gene (e.g., a CD47 gene) which is then transcribed under the control of regulatory elements contained in the viral long terminal repeat (LTR). A variety of single-gene-vector backbones have been used, including the Moloney murine leukemia virus (MoMuLV). Retroviral vectors which permit multiple insertions of different genes such as a gene for a selectable marker and a second gene of interest, under the control of an internal promoter can be derived from this type of backbone, see e.g., Gilboa, 1988, Adv. Exp. Med. Biol. 241:29.

The elements of the construction of vectors for the expression of a protein product are known to those skilled in the art. The most efficient expression from retroviral vectors is observed when “strong” promoters are used to control transcription, such as the SV 40 promoter or LTR promoters, reviewed in Chang et al., 1989, Int. J. Cell Cloning 7:264. These promoters are constitutive and do not generally permit tissue-specific expression. Other suitable promoters are discussed above.

The use of efficient packaging cell lines can increase both the efficiency and the spectrum of infectivity of the produced recombinant virions, see Miller, 1990, Human Gene Therapy 1:5. Murine retroviral vectors have been useful for transferring genes efficiently into murine embryonic, see e.g., Wagner et al., 1985, EMBO J. 4:663; Griedley et al., 1987 Trends Genet. 3:162, and hematopoietic stem cells, see e.g., Lemischka et al., 1986, Cell 45:917-927; Dick et al., 1986, Trends in Genetics 2:165-170.

One improvement in retroviral technology which permits attainment of much higher viral titers than were previously possible involves amplification by consecutive transfer between ecotropic and amphotropic packaging cell lines, the so-called “ping-pong” method, see e.g., Kozak et al., 1990, J. Virol. 64:3500-3508; Bodine et al., 1989, Prog. Clin. Biol. Res. 319:589-600.

Transduction efficiencies can be enhanced by pre-selection of infected marrow prior to introduction into recipients, enriching for those bone marrow cells expressing high levels of the selectable gene, see e.g., Dick et al., 1985, Cell 42:71-79; Keller et al., 1985, Nature 318:149-154. In addition, recent techniques for increasing viral titers permit the use of virus-containing supernatants rather than direct incubation with virus-producing cell lines to attain efficient transduction, see e.g., Bodine et al., 1989, Prog. Clin. Biol. Res. 319:589-600. Because replication of cellular DNA is required for integration of retroviral vectors into the host genome, it may be desirable to increase the frequency at which target stem cells which are actively cycling e.g., by inducing target cells to divide by treatment in vitro with growth factors, see e.g., Lemischka et al., 1986, Cell 45:917-927, a combination of IL-3 and IL-6 apparently being the most efficacious, see e.g., Bodine et al., 1989, Proc. Natl. Acad. Sci. 86:8897-8901, or to expose the recipient to 5-fluorouracil, see e.g., Mori et al., 1984, Jpn. J. Clin. Oncol. 14 Suppl. 1:457-463, prior to marrow harvest, see e.g., Lemischka et al., 1986, Cell 45:917-927; Chang et al., 1989, Int. J. Cell Cloning 7:264-280.

The inclusion of cytokines or other growth factors in the retroviral transformations can lead to more efficient transformation of target cells.

### Preparation of Transgenic Animals

Provided herein are cells, e.g., graftable cells, e.g., swine cells, e.g., hematopoietic stem cells, e.g., swine bone marrow cells, or other tissue which express a macrophage-inhibitory molecule (e.g., CD47) and, optionally, one or more additional molecules.

In particular, the recombinant swine cells are provided which express a human CD47 polypeptide, or a fragment thereof (e.g., a fragment that mediates inhibition of an immunological reaction, such as a macrophage-mediated reaction). The nucleotide sequence encoding the CD47 molecule can be part of a recombinant nucleic acid molecule that contains a tissue specific promoter located proximate to the human gene and regulating expression of the human gene in the swine cell. Tissues containing the recombinant sequence may be prepared by introducing a recombinant nucleic acid molecule into a tissue, such as bone marrow cells, using known transformation techniques. These transformation techniques include transfection and infection by retroviruses carrying either a marker gene or a drug resistance gene. See for example, Current Protocols in Molecular Biology, Ausubel et al. eds., John Wiley and Sons, New York (1987) and Friedmann (1989) Science 244:1275-1281. A tissue containing a recombinant nucleic acid molecule may then be reintroduced into an animal using reconstitution techniques (See for example, Dick et al. (1985) Cell 42:71). The present invention also includes swine, preferably miniature swine, expressing in its cells a recombinant CD47 nucleotide sequence. The recombinant constructs described above may be used to produce a transgenic pig by any method known in the art, including, but not limited to, microinjection, embryonic stem (ES) cell manipulation, electroporation, cell gun, transfection, transduction, retroviral infection, etc.

Transgenic animals (e.g., swine) can be produced by introducing transgenes into the germline of the animal. Embryonal target cells at various developmental stages can be used to introduce the human transgene construct. As is generally understood in the art, different methods are used to introduce the transgene depending on the stage of development of the embryonal target cell. One technique for transgenically altering an animal is to microinject a recombinant nucleic acid molecule into the male pronucleus of a fertilized egg so as to cause 1 or more copies of the recombinant nucleic acid molecule to be retained in the cells of the developing animal. The recombinant nucleic acid molecule of interest is isolated in a linear form with most of the sequences used for replication in bacteria removed. Linearization and removal of excess vector sequences results in a greater efficiency in production of transgenic mammals. See for example, Brinster et al. (1985) PNAS 82:4438-4442. In general, the zygote is the best target for micro-injection. In the swine, the male pronucleus reaches a size which allows reproducible injection of DNA solutions by standard microinjection techniques. Moreover, the use of zygotes as a target for gene transfer has a major advantage in that, in most cases, the injected DNA will be incorporated into the host genome before the first cleavage. Usually up to 40 percent of the animals developing from the injected eggs contain at least 1 copy of the recombinant nucleic acid molecule in their tissues. These transgenic animals will generally transmit the gene through the germ line to the next generation. The progeny of the transgenically manipulated embryos may be tested for the presence of the construct by Southern blot analysis of a segment of tissue. Typically, a small part of the tail is used for this purpose. The stable integration of the recombinant nucleic acid molecule into the genome of transgenic embryos allows permanent transgenic mammal lines carrying the recombinant nucleic acid molecule to be established.

Alternative methods for producing a mammal containing a recombinant nucleic acid molecule of the present invention include infection of fertilized eggs, embryo-derived stem cells, to potent embryonal carcinoma (EC) cells, or early cleavage embryos with viral expression vectors containing the recombinant nucleic acid molecule. (See for example, Palmiter et al. (1986) Ann. Rev. Genet. 20:465-499 and Capecchi (1989) Science 244:1288-1292)

Retroviral infection can also be used to introduce transgene into a cell. The developing embryo can be cultured in vitro to the blastocyst stage. During this time, the blastomeres can be targets for retroviral infection (Jaenich (1976) PNAS 73:1260-1264). Efficient infection of the blastomeres is obtained by enzymatic treatment to remove the zona pellucida (Hogan et al. (1986) in Manipulating the Mouse Embryo, Cold Spring Harbor Laboratory Press, Cold Spring Harbor, N.Y.). The viral vector system used to introduce the transgene is typically a replication-defective retrovirus carrying the transgene (Jahner et al. (1985) PNAS 82:6927-6931; Van der Putten et al. (1985) PNAS 82:6148-6152). Transfection can be obtained by culturing the blastomeres on a monolayer of virus-producing cells (Van der Putten, supra; Stewart et al. (1987) EMBO J. 6:383-388). Alternatively, infection can be performed at a later stage. Virus or virus-producing cells can be injected into the blastocoele (Jahner et al. (1982) Nature 298:623.628). Most of the founders will be mosaic for the transgene since incorporation typically occurs only in a subset of the cells which formed the transgenic swine. Further, the founder may contain various retroviral insertions of the transgene at different positions in the genome which generally will segregate in the offspring. In addition, it is also possible to introduce transgenes into the germ line, albeit with low efficiency, by intrauterine retroviral infection of the mid-gestation embryo (Jahner et al. (1982) supra).

A third approach, which may be useful in the construction of transgenic animals, would target transgene introduction into an embryonic stem cell (ES). ES cells are obtained from pre-implantation embryos cultured in vitro and fused with embryos (Evans et al. (1981) Nature 292:154-156; Bradley et al. (1984) Nature 309:255-258; Gossler et al. (1986) PNAS 83:9065-9069; and Robertson et al. (1986) Nature 322:445-448). Transgenes might be efficiently introduced into the ES cells by DNA transfection or by retrovirus-mediated transduction. Such transformed ES cells could thereafter be combined with blastocysts, e.g., from a swine. The ES cells could be used thereafter to colonize the embryo and contribute to the germ line of the resulting chimeric animal. For review, see Jaenisch (1988) Science 240:1468-1474.

Introduction of the recombinant gene at the fertilized oocyte stage ensures that the gene sequence will be present in all of the germ cells and somatic cells of the transgenic “founder” animal. As used herein, founder (abbreviated “F”) means the animal into which the recombinant gene was introduced at the one cell embryo stage. The presence of the recombinant gene sequence in the germ cells of the transgenic founder animal in turn means that approximately half of the founder animal's descendants will carry the activated recombinant gene sequence in all of their germ cells and somatic cells. Introduction of the recombinant gene sequence at a later embryonic stage might result in the gene's absence from some somatic cells of the founder animal, but the descendants of such an animal that inherit the gene will carry the activated recombinant gene in all of their germ cells and somatic cells.

### Microinjection of Swine Oocytes

In preferred embodiments the transgenic swine of the present invention is produced by: i) microinjecting a recombinant nucleic acid molecule into a fertilized swine egg to produce a genetically altered swine egg; ii) implanting the genetically altered swine egg into a host female swine; iii) maintaining the host female for a time period equal to a substantial portion of the gestation period of said swine fetus, iv) harvesting a transgenic swine having at least one swine cell that has developed from the genetically altered mammalian egg, which expresses the recombinant nucleic acid molecule.

In general, the use of microinjection protocols in transgenic animal production is typically divided into four main phases: (a) preparation of the animals; (b) recovery and maintenance in vitro of one or two-celled embryos; (c) microinjection of the embryos and (d) reimplantation of embryos into recipient females. The methods used for producing transgenic livestock, particularly swine, do not differ in principle from those used to produce transgenic mice. Compare, for example, Gordon et al. (1983) Methods in Enzymology 101:411, and Gordon et al. (1980) PNAS 77:7380 concerning, generally, transgenic mice with Hammer et al. (1985) Nature 315:680, Hammer et al. (1986) J Anim Sci 63:269-278, Wall et al. (1985) Biol Reprod. 32:645-651, Pursel et al. (1989) Science 244:1281-1288, Vize et al. (1988) J Cell Science 90:295-300, Muller et al. (1992) Gene 121:263-270, and Velander et al (1992) PNAS 89:12003-12007, each of which teach techniques for generating transgenic swine. See also, PCT Publication WO 90/03432, and PCT Publication WO 92/22646 and references cited therein.

One step of the preparatory phase comprises synchronizing the estrus cycle of at least the donor females, and inducing superovulation in the donor females prior to mating. Superovulation typically involves administering drugs at an appropriate stage of the estrus cycle to stimulate follicular development, followed by treatment with drugs to synchronize estrus and initiate ovulation. As described in the example below, pregnant mare's serum is typically used to mimic the follicle-stimulating hormone (FSH) in combination with human chorionic gonadotropin (hCG) to mimic luteinizing hormone (LH). The efficient induction of superovulation in swine depend, as is well known, on several variables including the age and weight of the females, and the dose and timing of the gonadotropin administration. See for example, Wall et al. (1985) Biol. Reprod. 32:645 describing superovulation of pigs. Superovulation increases the likelihood that a large number of healthy embryos will be available after mating, and further allows the practitioner to control the timing of experiments.

After mating, one or two-cell fertilized eggs from the superovulated females are harvested for microinjection. A variety of protocols useful in collecting eggs from pigs are known. For example, in one approach, oviducts of fertilized superovulated females can be surgically removed and isolated in a buffer solution/culture medium, and fertilized eggs expressed from the isolated oviductal tissues. See, Gordon et al. (1980) PNAS 77:7380; and Gordon et al. (1983) Methods in Enzymology 101:411. Alternatively, the oviducts can be cannulated and the fertilized eggs can be surgically collected from anesthetized animals by flushing with buffer solution/culture medium, thereby eliminating the need to sacrifice the animal. See Hammer et al. (1985) Nature 315:600. The timing of the embryo harvest after mating of the superovulated females can depend on the length of the fertilization process and the time required for adequate enlargement of the pronuclei. This temporal waiting period can range from, for example, up to 48 hours for larger breeds of swine. Fertilized eggs appropriate for microinjection, such as one-cell ova containing pronuclei, or two-cell embryos, can be readily identified under a dissecting microscope.

The equipment and reagents needed for microinjection of the isolated swine embryos are similar to that used for the mouse. See, for example, Gordon et al. (1983) Methods in Enzymology 101:411; and Gordon et al. (1980) PNAS 77:7380, describing equipment and reagents for microinjecting embryos. Briefly, fertilized eggs are positioned with an egg holder (fabricated from 1 mm glass tubing), which is attached to a micro-manipulator, which is in turn coordinated with a dissecting microscope optionally fitted with differential interference contrast optics. Where visualization of pronuclei is difficult because of optically dense cytoplasmic material, such as is generally the case with swine embryos, centrifugation of the embryos can be carried out without compromising embryo viability. Wall et al. (1985) Biol. Reprod. 32:645. Centrifugation will usually be necessary in this method. A recombinant nucleic acid molecule of the present invention is provided, typically in linearized form, by linearizing the recombinant nucleic acid molecule with at least 1 restriction endonuclease, with an end goal being removal of any prokaryotic sequences as well as any unnecessary flanking sequences. In addition, the recombinant nucleic acid molecule containing the tissue specific promoter and the sequence encoding the immune-inhibitory molecule may be isolated from the vector sequences using 1 or more restriction endonucleases. Techniques for manipulating and linearizing recombinant nucleic acid molecules are well known and include the techniques described in Molecular Cloning: A Laboratory Manual, Second Edition. Maniatis et al. eds., Cold Spring Harbor, N.Y. (1989).

The linearized recombinant nucleic acid molecule may be microinjected into the swine egg to produce a genetically altered mammalian egg using well known techniques. Typically, the linearized nucleic acid molecule is microinjected directly into the pronuclei of the fertilized eggs as has been described by Gordon et al. (1980) PNAS 77:7380-7384. This leads to the stable chromosomal integration of the recombinant nucleic acid molecule in a significant population of the surviving embryos. See for example, Brinster et al. (1985) PNAS 82:4438-4442 and Hammer et al. (1985) Nature 315:600-603. The microneedles used for injection, like the egg holder, can also be pulled from glass tubing. The tip of a microneedle is allowed to fill with plasmid suspension by capillary action. By microscopic visualization, the microneedle is then inserted into the pronucleus of a cell held by the egg holder, and plasmid suspension injected into the pronucleus. If injection is successful, the pronucleus will generally swell noticeably. The microneedle is then withdrawn, and cells which survive the microinjection (e.g. those which do not lysed) are subsequently used for implantation in a host female.

The genetically altered mammalian embryo is then transferred to the oviduct or uterine horns of the recipient. Microinjected embryos are collected in the implantation pipette, the pipette inserted into the surgically exposed oviduct of a recipient female, and the microinjected eggs expelled into the oviduct. After withdrawal of the implantation pipette, any surgical incision can be closed, and the embryos allowed to continue gestation in the foster mother. See, for example, Gordon et al. (1983) Methods in Enzymology 101:411; Gordon et al. (1980) PNAS 77:7390; Hammer et al. (1985) Nature 315:600; and Wall et al. (1985) Biol. Reprod. 32:645.

The host female mammals containing the implanted genetically altered mammalian eggs are maintained for a sufficient time period to give birth to a transgenic mammal having at least 1 cell, e.g. a bone marrow cell, e.g. a hematopoietic cell, which expresses the recombinant nucleic acid molecule of the present invention that has developed from the genetically altered mammalian egg.

At two-four weeks of age (post-natal), tail sections are taken from the piglets and digested with Proteinase K. DNA from the samples is phenol-chloroform extracted, then digested with various restriction enzymes. The DNA digests are electrophoresed on a Tris-borate gel, blotted on nitrocellulose, and hybridized with a probe consisting of the at least a portion of the coding region of the recombinant cDNA of interest which had been labeled by extension of random hexamers. Under conditions of high stringency, this probe should not hybridize with the endogenous pig gene, and will allow the identification of transgenic pigs.

For additional guidance and methods for producing transgenic swine, see Martin et al. Production of transgenic swine, Transgenic Animal Technology: A Laboratory Handbook, Carl A. Pinkert, ed., Academic Press; 315-388. 1994; U.S. Pat. No. 5,523,226; and U.S. Pat. No. 6,498,285.

The transgenic cells, organs, tissues, and animals described herein can include additional genetic modifications, such as modifications that render the cells and organs more suitable for xenotransplantation. Transgenic swine expressing inhibitors of complement are described, e.g., in U.S. Pat. No. 6,825,395. Compositions for depleting xenoreactive antibodies are described in U.S. Pat. No. 6,943,239. In some embodiments, the transgenic cells, organs, and animals further include transgenic nucleic acid molecules that direct the expression of enzymes, capable of modifying, either directly or indirectly, cell surface carbohydrate epitopes such that the carbohydrate epitopes are no longer recognized by natural antibodies in a host (e.g., a human host) or by the cell-mediated immune response of the host, thereby reducing the immune system response elicited by the presence of such carbohydrate epitopes. In various embodiments, the transgenic cells, organs and animals (e.g., non-human mammals such as swine) express nucleic acid molecules encoding functional recombinant α-Galactosidase A (αGalA) enzyme which modifies the carbohydrate epitope Galα(1,3)Gal. Such cells, organs, and animals are described in U.S. Pat. No. 6,455,037.

In various embodiments, the transgenic swine, and cells, tissues, and organs derived therefrom, is miniature swine which is at least partially inbred (e.g., the swine is homozygous at swine leukocyte antigen (SLA) loci, and/or is homozygous at at least 65%, 70%, 75%, 80%, 85%, 90%, 95%, or more, of all other genetic loci). See U.S. Pat. No. 6,469,229.

In various embodiments, the transgenic cells, organs, and animals described herein are deficient for expression of a carbohydrate-modifying enzyme, such that the cells, etc., are rendered less reactive to antibodies (e.g., natural antibodies) present in a xenogeneic host. Expression can be rendered deficient by inactivating a gene expressing the enzyme in an organism (e.g., using gene knockout technology, or by other methods such as RNA interference). Swine deficient for expression of one such carbohydrate modifying enzyme, α-1,3 galactosyltransferase, are described, e.g., in U.S. Pat. No. 6,849,448.

Transplantation

The compositions and methods described herein can be used as part of a transplantation (e.g., xenotransplantation) protocol. Treatments that promote tolerance and/or decrease immune recognition of transplanted cell, tissues, and organs include use of immunosuppressive agents (e.g., cyclosporine, FK506), antibodies (e.g., anti-T cell antibodies such as polyclonal anti-thymocyte antisera (ATG)), irradiation, and protocols to induce mixed chimerism. Various agents and regimens for inducing tolerance are described in U.S. Pat. Nos. 6,911,220; 6,306,651; 6,412,492; 6,514,513; 6,558,663; and 6,296,846. See also Kuwaki et al., Nature Med., 11(1):29-31, 2005, and Yamada et al., Nature Med. 11(1):32-34, 2005.

The organ can be any organ, e.g., a liver, e.g., a kidney, e.g., a heart. Implanted grafts may consist of organs such as liver, kidney, heart; body parts such as bone or skeletal matrix; tissue such as skin, intestines, endocrine glands; or progenitor stem cells of various types.

Natural antibodies can be eliminated by organ perfusion, and/or transplantation of tolerance-inducing bone marrow. Preparation of the recipient for transplantation, and maintenance of the recipient after transplantation, can include any or all of the following steps. Certain aspects described below are particularly useful for primate (e.g., human) recipients.

Recipients are treated with a preparation of horse anti-human thymocyte globulin (ATG) injected intravenously (e.g., at a dose of approx. 25-100 mg/kg, e.g., 50 mg/kg, e.g., at days −3, −2 , −1 prior to transplantation). The antibody preparation eliminates mature T cells and natural killer cells. The ATG preparation also eliminates natural killer (NK) cells. Anti-human ATG obtained from any mammalian host can also be used. In addition, if further T cell depletion is indicated, the recipient may be treated with a monoclonal anti-human T cell antibody, such as LoCD2b (Immerge BioTherapeutics, Inc., Cambridge, Mass.).

It may also be necessary or desirable to thymectomize and/or splenectomize the recipient. Thymic irradiation can be used (e.g., as an alternative to thymectomy).

The recipient can be administered low dose radiation in order to make room for newly injected bone marrow cells (if bone marrow is to be administered). A sublethal dose of between 100 rads and 400 rads whole body radiation, plus 700 rads of local thymic radiation (e.g., at day −1), has been found effective for this purpose.

The recipient can be treated with an agent that depletes complement, such as cobra venom factor (at approx. 5-10 mg/d, at days −1).

Natural antibodies can be absorbed from the recipient's blood by hemoperfusion of a liver of the donor species. Also, or alternatively, the cells, tissues, or organs used for transplantation may be genetically modified such that they are not recognized by natural antibodies of the host (e.g., the cells are α-1,3-galactosyltransferase deficient).

In some embodiments, maintenance therapy (e.g., beginning immediately prior to, and continuing for at least a few days after transplantation) includes treatment with a human anti-human CD154 mAb (e.g., ABI793, Novartis Pharma AG, Basel, Switzerland; ˜25 mg/kg). Mycophenolate mofetil (MMF; 25-110 mg/kd/d) may be administered to maintain whole blood levels to a desirable level. Methylprednisolone may also be administered, beginning on the day of transplantation, tapering thereafter over the next 3-4 weeks.

Various agents useful for supportive therapy (e.g., at days 0-14) include anti-inflammatory agents such as prostacyclin, dopamine, ganiclovir, levofloxacin, cimetidine, heparin, antithrombin, erythropoietin, and aspirin.

In some embodiments, donor stromal tissue is administered. Preferably it is obtained from fetal liver, thymus, and/or fetal spleen, may be implanted into the recipient, preferably in the kidney capsule. Thymic tissue can be prepared for transplantation by implantation under the autologous kidney capsule for revascularization. Stem cell engraftment and hematopoiesis across disparate species barriers is enhanced by providing a hematopoietic stromal environment from the donor species. The stromal matrix supplies species-specific factors that are required for interactions between hematopoietic cells and their stromal environment, such as hematopoietic growth factors, adhesion molecules, and their ligands.

As liver is the major site of hematopoiesis in the fetus, fetal liver can also serve as an alternative to bone marrow as a source of hematopoietic stem cells. The thymus is the major site of T cell maturation. Each organ includes an organ specific stromal matrix that can support differentiation of the respective undifferentiated stem cells implanted into the host. As an added precaution against graft-versus-host disease (GVHD), thymic stromal tissue can be irradiated prior to transplantation, e.g., irradiated at 1000 rads. As an alternative or an adjunct to implantation, fetal liver cells can be administered in fluid suspension.

Bone marrow cells (BMC), or another source of hematopoietic stem cells, e.g., a fetal liver suspension, of the donor can be injected into the recipient. Donor BMC home to appropriate sites of the recipient and grow contiguously with remaining host cells and proliferate, forming a chimeric lymphohematopoietic population. By this process, newly forming B cells (and the antibodies they produce) are exposed to donor antigens, so that the transplant will be recognized as self. Tolerance to the donor is also observed at the T cell level in animals in which hematopoietic stem cell, e.g., BMC, engraftment has been achieved. The use of xenogeneic donors allows the possibility of using bone marrow cells and organs from the same animal, or from genetically matched animals.

### EXAMPLES

### Example 1

Signal regulatory protein (SIRP)α is a critical immune inhibitory receptor on macrophages, and its interaction with CD47, a ligand for SIRPα, prevents autologous phagocytosis. It was examined whether interspecies incompatibility of CD47 contributes to the rejection of xenogeneic cells by macrophages. That data described below show that pig CD47 does not interact with mouse SIRPα. Similar to CD47−/− mouse cells, porcine red blood cells (RBCs) failed to induce SIRPα tyrosine phosphorylation in mouse macrophages. Blocking SIRPα with anti-mouse SIRPα mAb (P84) significantly enhanced the phagocytosis of CD47+/+ mouse cells, but did not affect the engulfment of porcine or CD47−/− mouse cells by mouse macrophages. CD47-deficient mice, whose macrophages do not phagocytose CD47−/− mouse cells, showed markedly delayed clearance of porcine RBCs compared to wild-type mouse recipients. Furthermore, mouse CD47 expression on porcine cells markedly reduced their phagocytosis by mouse macrophages both in vitro and in vivo. These results indicate that interspecies incompatibility of CD47 contributes to phagocytosis of xenogeneic cells by macrophages. Genetic manipulation of donor CD47 can improve its interaction with the recipient SIRPα and provides a novel approach to attenuate phagocyte-mediated xenograft rejection.

The severe shortage of allogeneic donors currently limits the number of organ transplants performed (Cooper et al., Annu Rev Medicine. 53:133-147, 2002). This supply-demand disparity can be corrected by the use of organs from other species (xenografts). In view of the ethical issues and impracticalities associated with the use of non-human primates, pigs are considered the most suitable organ donor species for humans. In addition to organ size and physiologic similarities to humans, the ability to rapidly breed and inbreed pigs makes them particularly amenable to genetic modifications that could improve their ability to function as organ donors to humans (Sachs, Path Biol. 42:217-219, 1994; Piedrahita and Mir, Am J Transplant 4 Suppl 6:43-50, 2004). However, xenotransplantation from pigs is hampered by immunologic rejection. In addition to the adaptive immune responses, which play critical roles in both allo- and xenograft rejection, the innate immune system also mediates strong rejection of organs and cells from discordant xenogeneic donors.

Studies in various models have shown that macrophages contribute significantly to xenograft rejection. In xenotransplantation recipients, macrophages are activated and recruited rapidly, and their responses to xenoantigens precede the activation of T cells (Fox et al., J Immunol. 166:2133, 2001). It has been reported that macrophages contribute significantly to the rejection of porcine hematopoietic cells (Abe et al., J Immunol. 168:621-628, 2002; Basker et al., Transplantation 72:1278-1285, 2001) and islet xenografts (Karlsson-Parra et al., Transplantation 61:1313-1320, 1996; Wu et al., Xenotransplantation. 2000; 7:214-220; Soderlund et al., Transplantation 67:784-791, 1999) in both rodents and primates. Similarly, macrophages also mediate strong rejection of human hematopoietic cells and islets in mice (Terpstra et al., Leukemia 11:1049-1054, 1997; Andres et al., Transplantation 79:543-549, 2005). The rapid and refractory rejection of xenogeneic hematopoietic cells by macrophages greatly impedes the application of mixed chimerism, a means of tolerance induction, to xenotransplantation.

Macrophage activation is regulated by the balance between activating and inhibitory signals. CD47 serves as a ligand for signal regulatory protein SIRPα, an immune inhibitory receptor on macrophages (Jiang et al., J Biol Chem. 274:559-562, 1999; Vernon-Wilson et al., Eur J Immunol. 30:2130-2137, 2000). Studies using CD47-deficient mice demonstrated that SIRPα on macrophages recognizes CD47 as a marker of “self” (Oldenborg et al., Science 288:2051-2054, 2000). CD47-SIRPα signaling prevents phagocytosis of normal hematopoietic cells by autologous macrophages and reduces the sensitivity of antibody- and complement-opsonized cells to phagocytosis (Oldenborg et al., Science 288:2051-2054, 2000; Blazar et al., J Exp Med. 194:541, 2001; Oldenborg et al., J Exp Med. 193:855-862, 2001; Oldenborg, Blood 99:3500-3504, 2002). These results indicate that macrophages rely on CD47 expression to distinguish “self” from “non-self” and to set a threshold for macrophage-mediated phagocytosis of opsonized cells. Thus, donor cells would be highly susceptible to phagocytosis by recipient macrophages in a xenogeneic transplantation setting if donor CD47 fails to interact with recipient SIRPα. To investigate this, the role of CD47 in phagocytosis of xenogeneic cells in the setting of pig-to-mouse xenotransplantation was examined. The results described below indicate that the failure of pig CD47 to interact with mouse SIRPα renders porcine cells highly sensitive to phagocytosis by mouse macrophages. Furthermore, genetic manipulation of donor CD47 to improve its interaction with the recipient SIRPα is effective in preventing the rejection of porcine cells by macrophages.

Results

Pig CD47 does not Interact with Mouse SIRPα

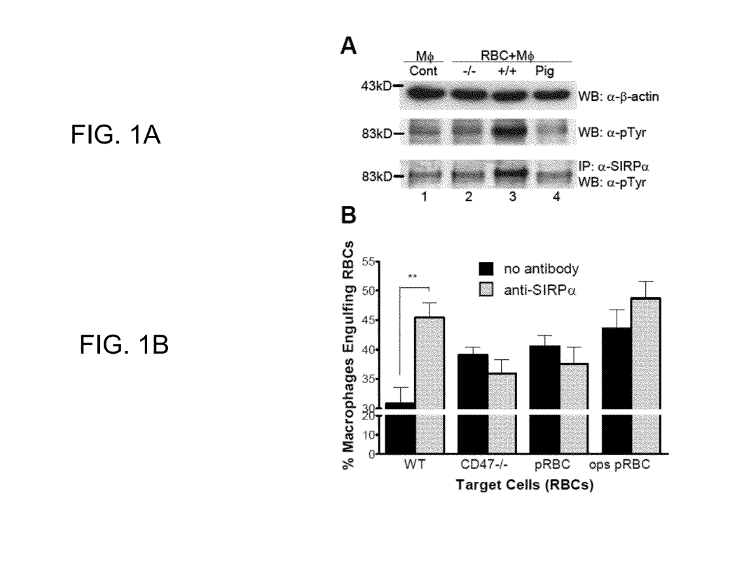

SIRPα contains intracellular immune receptor tyrosine-based inhibitory motifs (ITIMs). SIRPα activation after binding to CD47 results in tyrosine phosphorylation of ITIMs, leading to the recruitment and activation of protein tyrosine phosphatases (Kharitonenkov et al. Nature 386:181-186, 1997). To determine whether pig CD47 can interact with mouse SIRPα, SIRPα tyrosine phosphorylation was examined in bone marrow-derived macrophages after contact with porcine, CD47 knock-out (KO) and wild-type (WT) mouse RBCs. Western blot revealed that incubation of WT mouse macrophages with WT mouse RBCs resulted in significant SIRPα tyrosine phosphorylation (FIG. 1A, lane 3). However, similar to CD47 KO mouse RBCs, porcine RBCs failed to induce SIRPα tyrosine phosphorylation in WT mouse macrophages. Macrophages showed a similar low level of SIRPα tyrosine phosphorylation after incubation with CD47 KO mouse or porcine RBCs (FIG. 1A, lanes 2 and 4), or in medium alone (FIG. 1A, lane 1).

The effect of anti-mouse SIRPα blocking mAb (P84) on phagocytosis of porcine cells by mouse macrophages was examined using an in vitro phagocytic assay. Previous studies have shown that P84 blocks CD47-SIRPα interaction and thereby augments phagocytosis (Oldenborg et al., Science 288:2051-2054, 2000). P84 should not affect the phagocytosis of porcine RBCs by mouse macrophages if pig CD47 does not interact with murine SIRPα. In these experiments, WT mouse macrophages were incubated in medium with or without P84 for 20 min prior to the addition of target cells (i.e., CD47 KO mouse, WT mouse, and porcine RBCs). As shown in FIG. 1B, blocking SIRPα with P84 led to a significant increase in the engulfment of WT mouse RBCs, but had no effect on the higher baseline levels of ingestion of CD47 KO mouse or porcine RBCs (both untreated and antibody-opsonized) by WT mouse macrophages. Together, these results indicate that pig CD47 cannot deliver inhibitory signals to mouse macrophages through the SIRPα receptor.

Delayed Rejection of Porcine Cells in CD47 KO Compared to WT Mice

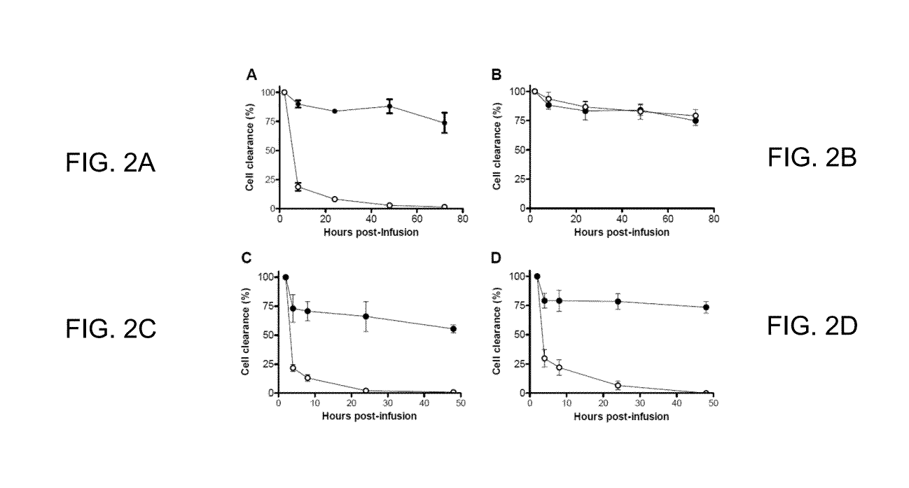

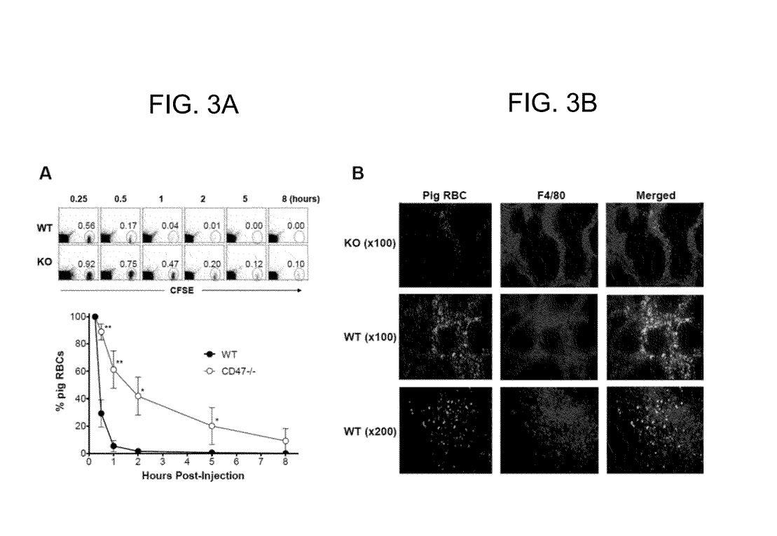

In CD47 KO mice, macrophages are adapted and do not phagocytose CD47−/− cells (Oldenborg et al., Science 288:2051-2054, 2000). CD47 KO cells were rapidly rejected after injection into syngeneic WT mice, but survived equivalently to WT mouse cells in CD47 KO mice (FIG. 2). Thus, it is expected that porcine cells will be more rapidly eliminated by macrophages in WT mice than in CD47 KO mice if pig CD47 cannot interact with mouse SIRPα. To address this question, the survival of porcine RBCs in WT and CD47 KO mice was compared. CFSE-labeled porcine RBCs were injected into WT or CD47 KO mice; blood was collected from the recipient mice at various times and the levels of injected porcine RBCs were measured by flow cytometric analysis. While porcine RBCs were completely rejected in both WT and CD47 KO mice, the clearance of porcine RBCs from blood was significantly delayed in CD47 KO mice. As shown in FIG. 3A, porcine cells were almost completely cleared from blood of WT mouse recipients by 2 hours, but remained detectable in CD47 KO mouse recipients 8 hours after cell transfer. Anti-pig xenoresponses by T cells, B cells, and NK cells may also contribute to the rejection of pig cells in the mouse recipients. However, the dramatic difference in the clearance of pig RBCs between WT and CD47 KO mice indicates that macrophages play an important role in the rejection of pig cells.

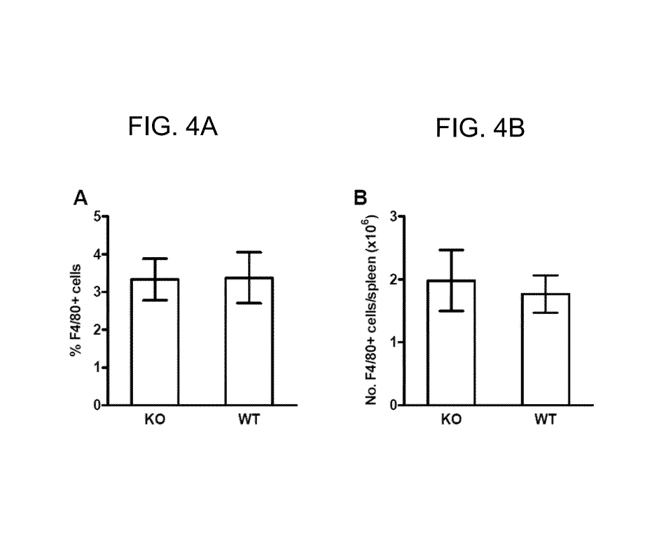

To further determine whether macrophages are responsible for the rapid clearance of porcine RBCs in WT recipients, frozen tissue sections were prepared from recipient spleens harvested 0.5, 1 and 2 hours after injection of CFSE-labeled porcine RBCs, and analyzed by fluorescence microscopy. Substantially greater numbers of porcine RBCs were detected in the red pulp area of WT compared to CD47 KO mouse recipients (FIG. 3B and data not shown). Immunofluorescence staining revealed that porcine cells detected in the red pulp were mainly engulfed by F4/80′ macrophages. Since WT and CD47 KO mice have a similar number of F4/80+ macrophages in the spleen (FIG. 3B and FIG. 4), these results suggest that the failure of pig CD47 to interact with mouse SIRPα may increase the susceptibility of porcine cells to phagocytosis by mouse macrophages.

Mouse CD47 Expression on Porcine Cells Reduces their Susceptibility to Phagocytosis by Mouse Macrophages

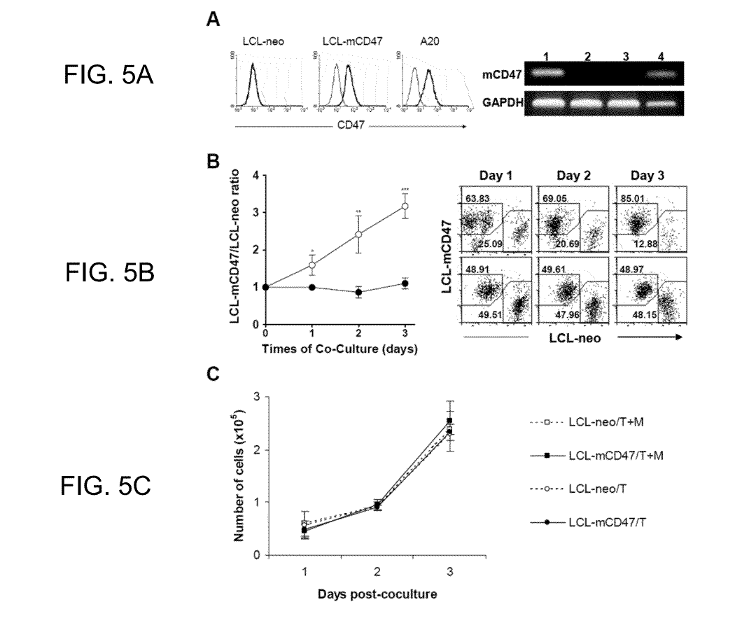

To further understand the role of CD47 in phagocytosis of xenogeneic cells and to determine whether expression of mouse CD47 on porcine cells could confer protection from phagocytosis by mouse macrophages, we generated mouse CD47-expressing (mCD47) porcine cell lines by transfection of porcine B lymphoma-like cells (LCL-13271) (Huang et al. Blood 97:1467-1473, 2001) with a mouse CD47 expressing plasmid (FIG. 5A). We compared the survival and expansion of mouse CD47 transfected (LCL-mCD47) and Neo transfected (control) (LCL-neo) porcine cells in cultures containing mouse macrophages. LCL-mCD47 and LCL-neo cells were labeled with different fluorescent dyes (red or green) and co-cultured at a 1:1 ratio in the presence and absence of mouse macrophages. The cultures were harvested daily for 3 days and the numbers of viable LCL-mCD47 and LCL-neo cells in the cultures were determined. As shown in FIG. 5B, the ratio of viable LCL-mCD47 to LCL-neo cells was significantly increased in the presence of mouse macrophages but remained constant in the absence of macrophages. However, in the transwell experiments, LCL-mCD47 and LCL-neo cells grew equally in the upper transwell chambers regardless of whether the lower chambers contained LCL target cells alone or along with mouse macrophages (FIG. 5C). These results imply that the increased expansion of LCL-mCD47 cells in the mixed cultures with mouse macrophages (FIG. 5B) reflects a mouse CD47-induced protection against direct contact-mediated cytotoxicity of mouse macrophages.

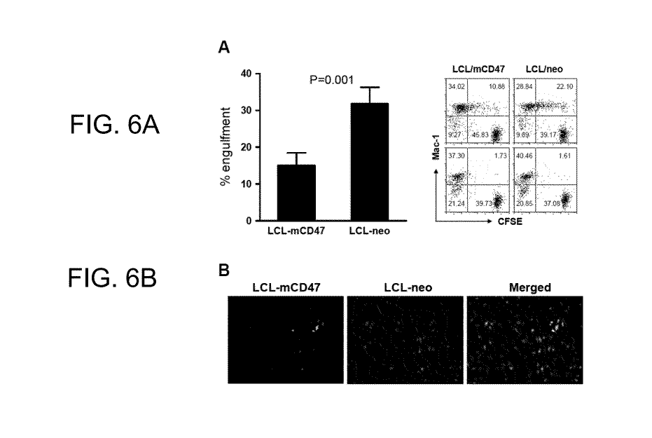

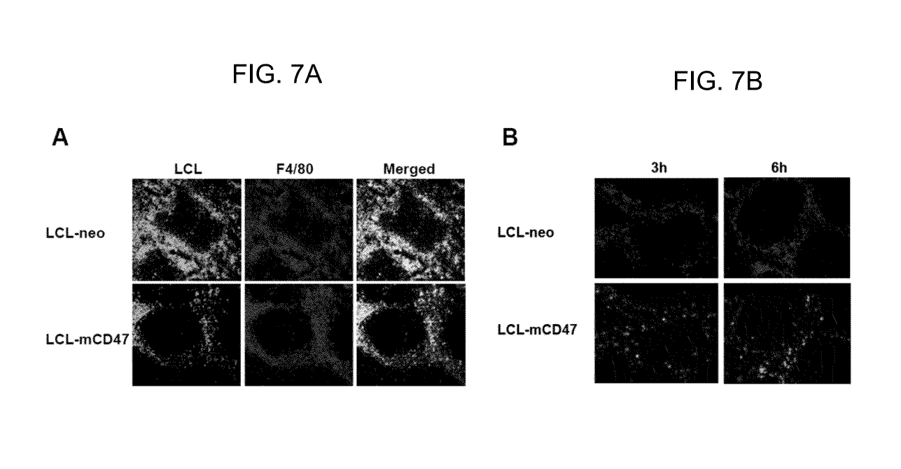

It was further confirmed that mouse CD47 expression on porcine cells prevents their phagocytosis by mouse macrophages. In in vitro phagocytic assays, mouse macrophages were markedly less effective in engulfing porcine LCL-mCD47 cells than engulfing LCL-neo cells (FIG. 6A). Mouse macrophages preferentially phagocytosed LCL-neo cells even when LCL-mCD47 and LCL-neo cells were both present, indicating that CD47 expression on individual target cells mediates this protection (FIG. 6B). The ability of mouse CD47 expression to prevent phagocytosis of porcine cells in vivo was assessed. Because red pulp macrophages in the spleen efficiently phagocytose CD47 KO mouse cells and porcine cells (FIG. 3), phagocytosis of CFSE-labeled LCL-mCD47 and LCL-neo cells in the mouse spleen was examined. More CFSE+ cells were detected in red pulp of the spleen in mice receiving LCL-neo cells than in those injected with LCL-mCD47 cells (FIG. 7A). Staining of mouse macrophages revealed that most porcine cells trapped in red pulp of the spleen were engulfed by macrophages (FIG. 7A). At 3 hours after cell infusion, almost all F4/80+ macrophages (stained red) in red pulp had engulfed porcine cells (appearing yellow in merged pictures) in mice injected with LCL-neo cells, whereas large numbers of red pulp macrophages showed no engulfment in mice that received LCL-mCD47 cells. Similar results were observed in mice injected with a mixture (1:1 ratio) of LCL-mCD47 and LCL-neo cells, in which more LCL-neo cells than LCL-mCD47 cells were detected in red pulp (i.e., engulfed by macrophages) (FIG. 7B).

Taken together, these results indicate that the lack of efficient interaction between pig CD47 and mouse SIRPα is an important factor contributing to the susceptibility of porcine cells to phagocytosis by mouse macrophages. Furthermore, mCD47 expression is effective in preventing the rejection of porcine cells by macrophages in mice.

Although macrophage depletion has been shown to be effective in preventing cellular xenograft rejection, the rapid recovery of macrophages and associated graft destruction after withdrawal of treatment indicates that sustained macrophage depletion or adaptation may be required to maintain long-term xenograft survival (Abe et al., J Immunol. 168:621-628, 2002; Terpstra et al., Leukemia 11:1049-1054, 1997; Andres et al., Transplantation 79:543-549, 2005; Fox et al., Transplantation 66:1407-1416, 1998). Because macrophages play a critical role in initiating immune responses against pathogens, strategies to specifically suppress xenogeneic cell-triggered macrophage activation are preferable to the long-term use of macrophage-depleting reagents. Such approaches can also be beneficial in solid organ xenotransplantation for which macrophages have also been implicated in rejection (Candinas et al., Transplantation 62:1920-1927, 1998; Wu et al., Xenotransplantation. 6:262-270, 1999).

The data described herein show that pig CD47 does not cross-react with mouse SIRPα. Ligation of the mouse SIRPα by mouse CD47 induces tyrosine phosphorylation of ITIMs (FIG. 1A), leading to the recruitment and activation of protein tyrosine phosphatases (Kharitonenkov et al. Nature 386:181-186, 1997). However, SIRPα phosphorylation could not be induced in mouse macrophages after incubation with porcine RBCs that express pig CD47 (FIG. 1A). Furthermore, blocking SIRPα with anti-mouse SIRPα mAb (P84) markedly augmented the engulfment of mouse cells, but did not affect the ingestion of porcine cells by mouse macrophages (FIG. 1B). To further understand the role of CD47 incompatibility in phagocytosis of xenogeneic cells, we established mouse CD47-expressing porcine cell lines. Both in vitro and in vivo phagocytic assays showed that forced expression of mouse CD47 on porcine cells can significantly reduce their susceptibility to phagocytosis by mouse macrophages (FIGS. 5-7). This shows that pig CD47 cannot deliver inhibitory signals to mouse macrophages via SIRPα, and that mouse CD47 expression prevents phagocytosis of porcine cells by mouse macrophages. These data indicate that CD47 is a molecular target for inhibiting macrophage-mediated rejection of xenogeneic cells.

The species specificity of CD47 has also been demonstrated in other species, and there is no evidence that a cross species CD47-SIRPα interaction can occur in a highly disparate xenogeneic combination (Vernon-Wilson et al., Eur J Immunol. 30:2130-2137, 2000; Okazawa et al., J Immunol. 174:2004-2011, 2005; Rebres et al., J Cell Physiol. 205:182-193, 2005). Human macrophages can phagocytose porcine cells in the absence of antibody or complement opsonization, and removing α1,3-galactosyl xenoantigens from porcine cells failed to prevent phagocytosis (Ide et al., Xenotransplantation 12:181-188, 2005). Considering the lack of cross reaction between CD47 and SIRPα in other species and the limited identity (73%) in amino acid sequences between pig and human CD47, the lack of cross-reaction between pig CD47 and human SIRPα is thought to be one mechanism resulting in phagocytosis of porcine cells by human macrophages (Shahein et al., Immunology 106:564-576, 2002).

Mixed hematopoietic chimerism has been shown to induce tolerance across the MHC barrier (Sykes, Immunity 14:417-424, 2001). Previous studies using a transgenic NOD/SCID mouse model suggested that mixed hematopoietic chimerism may also induce mouse and human T cell tolerance to porcine xenografts (Abe et al., Blood 99:3823-3829, 2002; Lan et al., Blood 103:3964-3969, 2004). However, unlike bone marrow transplantation within the same species, the innate immune system poses an obstacle to the establishment of donor hematopoiesis across discordant xenogeneic barriers (Yang, Springer Semin Immunopathol. 26:187-200, 2004). Macrophages mediate rejection of xenogeneic hematopoietic cells (Abe et al., J Immunol. 168:621-628, 2002; Basker et al., Transplantation 72:1278-1285, 2001). The rejection of porcine hematopoietic cells by host macrophages developing de novo in porcine hematopoietic chimeras suggests that mixed chimerism may not fully overcome the macrophage barrier. Therefore, inhibition of donor hematopoietic cell rejection by macrophages can promote xenotolerance induction through mixed chimerism. Studies in the CD47 KO mouse model have demonstrated that CD47 expression is critical for preventing phagocytosis of hematopoietic cells (Oldenborg et al., Science 288:2051-2054, 2000; Blazar et al., J Exp Med. 194:541, 2001). The rapid and vigorous rejection of CD47 KO hematopoietic cells in syngeneic WT mouse recipients suggests that CD47 incompatibility alone is sufficient to cause rejection of donor hematopoietic cells in a xenogeneic recipient. Thus, genetic manipulation of donor CD47 to improve its interaction with recipient SIRPα can promote donor hematopoietic engraftment and hence chimerism in xenogeneic recipients.

Although CD47-SIRPα interaction has been proven to be essential for the protection of normal hematopoietic cells from phagocytosis, it is unclear whether this interaction pathway also plays an important role in protecting non-hematopoietic tissues or cells from destruction by macrophages. Recent studies have shown that lung collectins, surfactant (SP)-A and SP-D, also bind SIRPα on alveolar macrophages through their globular heads to initiate an inhibitory signaling that helps to maintain a non- or anti-inflammatory lung environment (Gardai et al., Cell 115:13-23, 2003). These results suggest that the function of a porcine lung xenograft could also be severely compromised if porcine surfactants cannot bind human SIRPα. Among the other immune inhibitory receptors on macrophages, CD200 receptor (CD200R, also known as OX2R) has been shown to play a critical role in the regulation of tissue macrophage activation. The ligand for CD200R, CD200 (also known as OX2), is widely expressed throughout the body. Studies using CD200-deficient mice demonstrated that the absence of CD200-CD200R signaling leads to accelerated activation and expansion of tissue macrophages (Hoek et al., Science 290:1768-1771, 2000; Wright et al., Immunity 13:233-242, 2000). In addition to SIRPα and CD200R, paired Ig-like receptor (PIR)-B, immunoglobulin-like transcript (ILT) 3, and CD33-related receptors have also been shown to serve as inhibitory receptors for macrophages (Nakamura et al., Nat Immunol. 5:623-629, 2004; Cella et al., J Exp Med. 185:1743-1751, 1997; Crocker et al., Trends in Immunology 22:337-342, 2001). Considering the possibility of functional overlap (or redundancy) among these receptors in the normal situation, macrophages may mediate more robust phagocytosis of xenogeneic cells if the donor and host are incompatible for multiple immune inhibitory receptor-ligand interactions. In this regard, identifying the cross-reactivity of the major macrophage inhibitory receptors between pigs and humans facilitates understanding and manipulation of the robust xenoreactivity of macrophages, and provides approaches for attenuating macrophage mediated xenograft rejection.

Materials and Methods

Animals.

C57BL/6 (B6) mice were purchased from the Jackson Laboratories (Bar Harbor, Me.); CD47 gene knockout (CD47 KO) mice on a B6 background were generated as previously described (Oldenborg et al., Science 288:2051-2054, 2000). We used inbred Massachusetts General Hospital miniature swine (kindly provided by Dr. David H. Sachs) as porcine cell donors. Care of animals was in accordance with the Guide for the Care and Use of Laboratory Animals prepared by the National Academy of Sciences and published by the National Institutes of Health. Protocols involving animals were approved by the Massachusetts General Hospital Subcommittee on Research Animal Care.

Antibodies.

An anti-SIRPα antibody (P84) (Jiang et al., J Biol Chem. 274:559-562, 1999) was used to block macrophage inhibitory receptor SIRPα. Fluorescein isothiocyanate (FITC)-conjugated anti-mouse CD47 (miap 301, Pharmingen) and R-phycoerythrin (R-PE) conjugated anti-F4/80 (Caltag Laboratories, Burlingame, Calif.) were used for flow cytometry and immunohistology. In flow cytometric analyses, nonspecific binding of labeled mAbs was blocked with 2.4G2 (rat anti-mouse FCγR mAb); HOPC1 (murine IgG2a) and rat IgG (both from Pharmingen) were used as isotype controls.

Mouse Macrophage Preparation.

Bone marrow-derived and splenic macrophages were prepared as previously described (Oldenborg et al., Science 288:2051-2054, 2000; Oldenborg et al., J Exp Med. 193:855-862, 2001). To prepare peritoneal macrophages, peritoneal cells were harvested from B6 mice 4 days after intraperitoneal injection of 2% of Bio-Gel polyacrylamide P 100 (1 mL/mouse; Bio-RAD Laboratories Hercules, Calif.) and cultured at 37° C. for 2 hrs. Macrophages were used after washing off the nonadherent cells.

Immunoprecipitation and Western Blot Analysis.

Bone marrow-derived macrophages (2×10⁶) were plated on 150×25 mm plastic Petri dishes (Becton Dickinson, Franklin Lakes, N.J.) for 16 hours and then rinsed once with PBS prior to plating of mouse or porcine RBCs. The cultures were kept in a 37° C. water bath for 30 min. After lysing RBCs in cold ACK lysing buffer (Cambrex Bio Science Walkersville, Inc. Walkersville, Md.), macrophages were harvested, washed with PBS, and lysed in 0.4 ml of lysis buffer [50 mM Tris-HCl (pH 7.5), 150 mM NaCl, 1% NP-40, 1 mM phenylmethylsulfonyl fluoride (PMSF), 1% protease inhibitor cocktail (Sigma) and 2 mM sodium pervanadate]. The whole-cell lysates were assayed for protein quantity, using a Bio-Rad protein assay kit. For Western blot, 30 μg of macrophage lysates were separated on 10% SDS-PAGE and blotted onto nitrocellulose membrane. The membrane was stained with mouse anti-actin mAb IgG (C-2; Upstate, Charlottesville Va.) followed by bovine anti-mouse IgG-HRP (Upstate), or with rabbit anti-phosphotyrosine IgG (Upstate) followed by goat anti-rabbit IgG-HRP (Santa Cruz Biotechnology, Inc., Santa Cruz, Calif.). For immunoprecipitation, 300 μg of macrophage lysates were mixed with rat anti-mouse SIRPα antibody (P84) and a 50% slurry of protein G-Sepharose beads (Sigma) by rotation at 4° C. for 2 hrs. Precipitated proteins were separated on 10% SDS-PAGE, transferred to nitrocellulose membrane for Western blotting, in which rabbit immunoaffinity purified anti-phosphotyrosine IgG (Upstate) and goat anti-rabbit HRP-conjugated IgG (Santa Cruz Biotechnology, Inc.) were used as primary and secondary antibodies, respectively.

Mouse CD47 cDNA Plasmid Construction and Transfection.

Mouse CD47 expressing plasmid (pCDNA3.1-mCD47) was prepared by inserting full-length mouse CD47 cDNA (kindly provided to us by Dr. Tadashi Furusawa, National Institute of Animal Research Industry, Japan) into a eukaryotic expression vector pCDNA-3.1 (Invitrogen, Carlsbad, Calif.). LCL-13271 cells (a pig lymphoma-like cell line kindly provided by Dr. Christene Huang) (Sharland et al., Transplantation 76:1615-1622, 2003) were transfected with pCDNA3.1-mCD47 or the empty plasmid (pCDNA3.1-neo) using the Effectene Transfection kit (Qiagen Inc., Valencia, Calif.), and selected by incubation with 0.8 mg/mL of G418 (Gibco, Carlsbad, Calif.).

In Vitro Phagocytic Assay.

Fluorescent labeling of cells with green fluorescent dye carboxyfluorescein diacetate succinimidyl ester (referred to as CFSE), red fluorescent dye PKH-26-GL (referred to as PKH-26), and blue fluorescent dye 7-amino-4-chloromethylcoumarin (referred to as CMAC) was performed according to the manufacturer's protocols (Molecular Probes, Eugene, Oreg.). Fluorescent dye (CFSE or PKH-26)-labeled target cells were incubated with splenic or peritoneal macrophages. The cultures were harvested at various times and analyzed for numbers of viable target cells and phagocytosis by flow cytometry. The numbers of viable target cells were calculated as the product of the total number of viable cells (as counted by trypan blue exclusion) and the percentage of target cells (as measured by flow cytometry). To measure phagocytosis, CFSE-labeled target cells were incubated with macrophages; the cells were harvested at the indicated times and stained with anti-mouse Mac-1-PE prior to flow cytometric analysis. Phagocytosis was also measured using fluorescence microscopy, in which target cells and macrophages were labeled with different fluorescent colors. At the indicated times after incubation, non-ingested target cells were washed off, or for RBCs, were lysed with ACK buffer, and wells were viewed under a Nikon Eclipse TE2000-U fluorescent microscope.

Transwell Experiments.

These experiments were performed using 24-well plates with transwell inserts (0.4-μm pore size, Costar Inc., Cambridge, Mass.). A mixture (1:1 ratio) of unlabeled LCL-mCD47 and LCL-neo cells (1×10⁵/well) was added to the lower chamber with or without mouse macrophages (1×10⁶/well), and a mixture (1:1 ratio) of LCL-mCD47 and LCL-neo cells (1×10⁵/well) labeled with different fluorescent colors (CFSE or PKH-26) was placed in the upper transwell chamber. The plates were then incubated at 37° C. At various times after incubation, the cultures were harvested and the numbers of LCL-mCD47 and LCL-neo porcine cells in the upper transwell chambers were determined by flow cytometry as described in the in vitro phagocytic assay above.

RBC Clearance Assay.

The assay was performed as previously described (Oldenborg et al., Science 288:2051-2054, 2000). Briefly, fresh pig RBCs were labeled with CFSE and injected (i.v.) into WT or CD47 KO mice (2×10⁸ RBCs per mouse). RBC clearance was measured by flow cytometric analysis of 5 μL blood samples collected at various times. In some experiments, recipient spleens were harvested at various times after pig RBC injection and stored at −70° C. Frozen sections (8 μm) were prepared, fixed in acetone for 10 min at 4° C., and stained with PE-labeled rat anti-mouse F4/80 (Caltag Laboratories) overnight at 4° C. After being washed and mounted, slides were viewed under a Nikon Eclipse TE2000 fluorescent microscope.

In Vivo Phagocytic Assay.

CFSE-labeled target cells were injected (i.v.) into mice. The recipient spleens were harvested at various times and stored at −70° C. Frozen sections were prepared, fixed in acetone for 10 min at 4° C., and stained with PE-labeled rat anti-mouse F4/80 (Caltag Laboratories) overnight at 4° C. After being washed and mounted, slides were viewed under a Nikon Eclipse TE2000-U fluorescent microscope.

Statistical Analysis.

Significant differences between groups were determined using the Student's t test. A P value of less than 0.05 was considered statistically significant.

### Example 2

Human macrophages phagocytose porcine cells in the absence of antibody or complement opsonization, and that the removal of α1,3-galactosyl xenoantigens from the porcine cells failed to prevent the phagocytosis. SIRPα is a critical immune inhibitory receptor on macrophages, and its interaction with CD47, a ligand for SIRPα, prevents autologous phagocytosis. Considering the limited compatibility (73%) in amino acid sequences between pig and human CD47, it was hypothesized that the interspecies incompatibility of CD47 may contribute to the rejection of xenogeneic cells by macrophages.

In order to determine whether pig CD47 interacts with human SIRPα, SIRPα tyrosine phosphorylation in human macrophages after contact with porcine and human RBCs was compared. To further determine whether the expression of human CD47 on porcine cells confers protection from phagocytosis by human macrophages, human CD47-expressing porcine cell lines were generated by transfecting porcine B lymphoma-like cells (LCL) with a human CD47 expressing plasmid. The phagocytotic activities of human macrophages toward porcine LCL were evaluated by in vitro assays in the presence or absence of anti-porcine antibodies and complement. Briefly, carboxyfluorescein succinimidyl ester (CFSE)-labeled human CD47-transfected LCL (LCL-hCD47) and control vector-transfected LCL (LCL-pKS336) were incubated with human peripheral and reticuloendothelial macrophages (Kupffer cells) for 4 h in the presence or absence of human interferon (IFN)-γ.

Results

Western blotting revealed that the incubation of human macrophages with human RBCs resulted in significant SIRPα tyrosine phosphorylation. However, SIRPα tyrosine phosphorylation was not induced in human macrophages incubated with porcine RBCs. Macrophages incubated with medium alone also did not exhibit SIRPα phosphorylation. Human CD47 expression on porcine cells radically reduced the susceptibility of the cells to phagocytosis by human peripheral and reticuloendothelial macrophages, regardless of the presence or absence of antibody opsonization.

These results indicate that the interspecies incompatibility of CD47 significantly contributes to the rejection of xenogeneic cells by macrophages. Genetic manipulation of porcine cells for expression of human CD47 provides a novel approach to attenuating macrophage-mediated xenograft rejection through inhibitory CD47-SIRPα signaling.

A number of embodiments of the invention have been described. Nevertheless, it will be understood that various modifications may be made without departing from the spirit and scope of the invention. Accordingly, other embodiments are within the scope of the following claims.

What is claimed is:

1. A cell of a first species comprising a nucleotide sequence encoding a CD47 polypeptide, or fragment thereof, of a second species.

2. The cell of claim 1, wherein the first species is a non-human mammalian species.

3. The cell of claim 1, wherein the first species is a swine species.

4. The cell of claim 1, wherein the second species is human.

5. The cell of claim 1, further comprising a second nucleotide sequence encoding a second polypeptide of the second species.

6. The cell of claim 1, wherein the cell is deficient for expression of a carbohydrate modifying enzyme.

7. The cell of claim 1, which is a hematopoietic cell.

8. A transgenic non-human mammal whose genome comprises a nucleotide sequence encoding a human CD47 polypeptide.

9. The mammal of claim 8, wherein the mammal is a swine.

10. An organ from the transgenic mammal of claim 8.

11. The organ of claim 10, wherein the first species is a non-human mammalian species.

12. The organ of claim 11, wherein the first species is a swine species.

13. The organ of claim 10, wherein the second species is human.

14. The organ of claim 10, wherein the mammal further comprises a second nucleotide sequence encoding a polypeptide of the second mammalian species.

15. The organ of claim 10, wherein the mammal is deficient for expression of a carbohydrate modifying enzyme.

16. A method of supplying a graft, comprising:
providing a donor graft, wherein said graft expresses a heterologous CD47 polypeptide or over express an endogenous CD47 polypeptide;
implanting said graft in a recipient;
thereby supplying a graft.

17. The method of claim 16, wherein said donor and recipient are of different species.

18. The method of claim 16, wherein said donor and recipient are of same species and expression of CD47 on the graft is upregulated.


SEQUENCE LISTING (6 sequences)

SEQ ID NO: 1 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 2 - protein, 304 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRN

SEQ ID NO: 3 - protein, 312 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNE

SEQ ID NO: 4 - DNA, 5346 bp, Homo sapiens
ggggagcaggcgggggagcgggcgggaagcagtgggagcgcgcgtgcgcgcggccgtgcagcctgggcagtgggtcctgcctgtgacgcgcggcggcggtcggtcctgcctgtaacggcggcggcggctgctgctccagacacctgcggcggcggcggcgaccccgcggcgggcgcggagatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgcattcaaagaatcaaaaggaatgatgaatgatgaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctattttgttgggctatttctattgctgctacagcagaccacaagcacatttctgaaaaatttaatttattaatgtatttttaagttgcttatattctaggtaacaatgtaaagaatgatttaaaatattaattatgaattttttgagtataatacccaataagcttttaattagagcagagttttaattaaaagttttaaatcagtc

SEQ ID NO: 5 - DNA, 5288 bp, Homo sapiens
ggggagcaggcgggggagcgggcgggaagcagtgggagcgcgcgtgcgcgcggccgtgcagcctgggcagtgggtcctgcctgtgacgcgcggcggcggtcggtcctgcctgtaacggcggcggcggctgctgctccagacacctgcggcggcggcggcgaccccgcggcgggcgcggagatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctattttgttgggctatttctattgctgctacagcagaccacaagcacatttctgaaaaatttaatttattaatgtatttttaagttgcttatattctaggtaacaatgtaaagaatgatttaaaatattaattatgaattttttgagtataatacccaataagcttttaattagagcagagttttaattaaaagttttaaatcagtc

SEQ ID NO: 6 - DNA, 5313 bp, Homo sapiens
ggggagcaggcgggggagcgggcgggaagcagtgggagcgcgcgtgcgcgcggccgtgcagcctgggcagtgggtcctgcctgtgacgcgcggcggcggtcggtcctgcctgtaacggcggcggcggctgctgctccagacacctgcggcggcggcggcgaccccgcggcgggcgcggagatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctattttgttgggctatttctattgctgctacagcagaccacaagcacatttctgaaaaatttaatttattaatgtatttttaagttgcttatattctaggtaacaatgtaaagaatgatttaaaatattaattatgaattttttgagtataatacccaataagcttttaattagagcagagttttaattaaaagttttaaatcagtc